SEQUENCE LISTING (16 sequences)

SEQ ID NO: 1 - DNA, 1341 bp, Homo sapiens
caagttcagctggtggagtctggaggaggagtagtacaacctggaaggtcactgagactgtcttgtaaggctagtggattcaatatcaaggactattatatgcactgggtcagacaagctcctggaaaaggactcgagtggataggtttaattgatcctgagaatggtaacacgatatatgatcccaagttccaaggaagattcacaatttctgcagacaactctaagaatacactgttcctgcagatggactcactcagacctgaggatacagcagtctactattgtgctagagataacagttattacttcgactactggggccaaggaacaccagtcaccgtgagctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 2 - protein, 447 aa, Artificial Sequence
  Therapeutic antibodies comprised of Fc-variants of human monoclonal antibody CNTO 860 and equivalent antibodies with human heavy chain andhuman light chain variable amino acids
QVQLVESGGGVVQPGRSLRLSCKASGFNIKDYYMHWVRQAPGKGLEWIGLIDPENGNTIYDPKFQGRFTISADNSKNTLFLQMDSLRPEDTAVYYCARDNSYYFDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPXVFLFPPKPKDTLMISRTPEVTCVVXDVSHEDPEVKFNWYVDGVEVHNAKTKPREEQYNSTYRVVSVLTVLHQDWLNGKEYKCKVSNKALPXPXEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - DNA, 645 bp, Homo sapiens
gatatccaaatgacacaatctccttcttctctaagtgcttctgtcggagatagagtaacaattacatgtaaggcgagtcaggacattagaaagtatttaaactggtatcagcaaaaacctgggaaggctcctaagctactgatttattatgcaacaagtttggcagatggagtaccttctagattttctggttctggctctggaacagactacacattcacaatttcttctctccaacctgaggacattgctacatactactgcctacaacatggtgagagtccgtatacatttggacaaggaacaaaactagagatcacacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 4 - protein, 214 aa, Artificial Sequence
  Therapeutic antibodies comprised of Fc-variants of human monoclonal antibody CNTO 860 and equivalent antibodies with human heavy chain andhuman light chain variable amino acids
DIQMTQSPSSLSASVGDRVTITCKASQDIRKYLNWYQQKPGKAPKLLIYYATSLADGVPSRFSGSGSGTDYTFTISSLQPEDIATYYCLQHGESPYTFGQGTKLEITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - DNA, 43 bp, Homo Sapiens
caaagccctcccagctccggaggagaaaaccatctccaaagcc

SEQ ID NO: 6 - DNA, 43 bp, Homo sapiens
ggctttggagatggttttctcctccggagctgggagggctttg

SEQ ID NO: 7 - DNA, 39 bp, Homo sapiens
caacaaagccctcccaatcccgatcgagaaaaccatctc

SEQ ID NO: 8 - DNA, 39 bp, Homo sapiens
gagatggttttctcgatcgggattgggagggctttgttg

SEQ ID NO: 9 - DNA, 39 bp, Homo sapiens
caacaaagccctcccatacccgatcgagaaaaccatctc

SEQ ID NO: 10 - DNA, 39 bp, Homo sapiens
gagctggttttctcgagcgggtatgggagggctttgttg

SEQ ID NO: 11 - DNA, 38 bp, Homo sapiens
ccaacaaagccctcccaatcccggaggagaaaaccatc

SEQ ID NO: 12 - DNA, 38 bp, Homo sapiens
gatggttttctcctccgggattgggagggctttgttgg

SEQ ID NO: 13 - DNA, 40 bp, Homo sapiens
gaggtcacatgcgtggtcatcgatgtgagccacgaagacc

SEQ ID NO: 14 - DNA, 40 bp, Homo sapiens
ggtcttcgtggctcacatcgatgaccacgcatgtgacctc

SEQ ID NO: 15 - DNA, 33 bp, Homo sapiens
ctcctggggggtccggacgtcttcctcttcccc

SEQ ID NO: 16 - DNA, 33 bp, Homo sapiens
ggggaagaggaagacgtccggaccccccaggag

(19) United States
(12) Patent Application Publication
(10) Pub. No.: US 2008/0311034 A1
(43) Pub. Date: Dec. 18, 2008

(54) Anti-Tissue Factor Antibodies and Compositions with Enhanced Effector Function

(76) Inventors: Glenn M. Anderson, Radnor, PA (US); Bernard Scallon, Radnor, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/110,653

(22) Filed: Apr. 28, 2008

Related U.S. Application Data
(60) Provisional application No. 60/914,882, filed on Apr. 30, 2007.

(51) Int. Cl. A61K 39/395; C07K 16/28; A61K 51/10; C07H 21/00; C12N 5/16; A01K 67/027; A61P 9/00; A61P 31/00; A61P 37/00

(52) U.S. Cl. 424/1.49; 530/389.1; 530/388.1; 424/173.1; 424/178.1; 536/23.53; 435/326; 800/13

(57) ABSTRACT

Variants of antibodies to human tissue factor are optimized for the capability to elicit effector functions produced by immune effector cells while maintaining the ability to neutralize the biological functions of tissue factor, such as the prevention of blood clotting via the extrinsic pathway, and are produced by mutations of the Fc-region of the antibody. The variants comprise A330Y, A330I, and I332E where the I332E variant may optionally further comprise a second substitution selected from A330I (in the case of the first substitution being I332E), V264I, and S239D. The variants may further be optimized for the capability to elicit effector functions produced by immune effector cells by production of the antibody variants under conditions that produce effector function enhancing glycosylation of the Fc-region.

### CROSS-REFERENCE TO RELATED APPLICATIONS

This application claims the benefit of U.S. Provisional Application Ser. No. 60/914,882, filed 30 Apr. 2007, the entire contents of which are incorporated herein by reference in its entirety.

### FIELD OF THE INVENTION

The present invention relates to antibodies which bind to human tissue factor, including specified portions or variants thereof. The antibodies of the invention have the ability to interact with effector cells to activate innate immunity in addition to their human tissue factor neutralizing activity and are thus particularly useful in methods for treating tumor cells. The invention also relates to nucleic acids encoding such anti-tissue factor antibodies, complementary nucleic acids, vectors, host cells, and methods of making and using thereof, including therapeutic formulations, administration and devices and, more particularly, antibodies with enhanced effector function.

### BACKGROUND OF THE INVENTION

The coagulation of blood involves a cascading series of reactions leading to the formation of fibrin. The coagulation cascade consists of two overlapping pathways, both of which are required for hemostasis. The intrinsic pathway comprises protein factors present in circulating blood, while the extrinsic pathway requires tissue factor (TF), which is expressed on the cell surface of a variety of tissues in response to vascular injury (Davie et al., 1991, Biochemistry 30:10363). When exposed to blood, TF sets in motion a potentially explosive cascade of activation steps that result in the formation of an insoluble fibrin clot. TF has been investigated as a target for anticoagulant therapy.

TF is a single chain, 263-amino acid membrane glycoprotein that functions as a receptor for factor VII and VIIa and thereby initiates the extrinsic pathway of the coagulation cascade in response to vascular injury. TF, a transmembrane cell surface receptor, serves as the receptor as well as the cofactor for factor VIIa, forming a proteolytically active TF:VIIa complex on cell surfaces (Ruf et al, (1992) J. Biol. Chem. 267:6375-6381). In addition to its role in maintaining hemostasis, excess TF has been implicated in pathogenic conditions. Specifically, the synthesis and cell surface expression of TF has been implicated in vascular disease (Wilcox et al., 1989, Proc. Natl. Acad. Sci, 86:2839) and gram-negative septic shock (Warr et al., 1990, Blood 75:1481). Recent evidence suggests that disulfide isomerization of the TF extracellular domain may control functional properties of TF on the cell surface. Ahamed, J., Versteeg, H. H., Kerver, M., Chen, V. M., Mueller, B. M., Hogg, P. J. and Ruf, W. 2006. “Disulfide isomerization switches tissue factor from coagulation to cell signaling” Proc. Natl. Acad. Sci. U.S.A. 103 (38), 13932-13937).

Tissue factor is also overexpressed on a variety of malignant tumors and isolated human tumor cell lines, suggesting a role in tumor growth and survival. TF is not produced by healthy endothelial cells lining normal blood vessels but is expressed on these cells in tumor vessels. Aberrant expression of TF on endothelial and tumor cells in a variety of breast, colorectal, lung and pancreatic cancers has been linked to an increase in tumor microvessel density and upregulated VEGF expression. Tumor cells over expressing TF are also thought to be responsible for the thrombotic complications associated with cancer. Thus, there is a rationale for the inhibition of tissue factor in the treatment of cancer.

Naturally occurring and man-made antagonists of TF exist. Tissue factor pathway inhibitor proteins, TFPI-1 (NP_{—}001027452) and TFPI-2 (NP_{—}006519), are protease inhibitors that regulate the tissue factor (TF)-dependent pathway of blood coagulation. Various anti-TF antibodies are known capable of neutralizing biological functions of TF (Carson et al, (1987, Blood 70:490-493; Ruf et al. 1991. Thrombosis and Haemostasis 66:529). One monoclonal antibody, TF8-5G9, capable of inhibiting the TF/VIIa complex, is disclosed in U.S. Pat. Nos. 6,001,978; 5,223,427; and 5,110,730. Ruf et al. suggested (supra) that mechanisms that inactivate the TF/VIIa complex, rather than prevent its formation, may provide strategies for interruption of coagulation in vivo. WO 96/40921 discloses CDR-grafted anti-TF antibodies derived from the TF8-5G9 antibody. Other humanized or human anti-TF antibodies are disclosed in, e.g., Presta et al, Thromb Haemost 85:379-389 (2001), EP1069185, WO 01/70984 and WO03/029295.

The Fc region of an antibody interacts with a number of Fc receptors and ligands, imparting an array of important functional capabilities referred to as effector functions. For IgG the Fc region comprises Ig CH2 and CH3 (also called Cy2 and Cy3) domains of the heavy chain as well as the CH1 domain and the hinge region. An important family of Fc receptors for the IgG class are the Fc gamma receptors (FcγRs) which allow the cells bearing such receptors to effect responses. Thus, the FcγR constitutes a bridge between the humoral (antigen binding function) and the cellular arm of the immune system (Raghavan et al., 1996, Annu Rev Ce!! Dev Biol 12:181-220; Ravetch et al., 2001, Annu Rev Immunol 19:275-290; both expressly incorporated by reference). These receptors are expressed in a variety of immune cells including monocytes, macrophages, neutrophils, dendritic cells, eosinophils, mast cells, platelets, B cells, large granular lymphocytes, Langerhans' cells, natural killer (NK) cells, and T cells. Formation of the Fc/FcγR complex recruits these “effector cells” to sites of bound antigen, typically resulting in signaling events within the cells and important subsequent immune responses, such as release of inflammation mediators, B cell activation, endocytosis, phagocytosis, and cytotoxic attack. The ability to mediate cytotoxic and phagocytic effector functions is a potential mechanism by which antibodies destroy targeted cells. The cell-mediated reaction wherein nonspecific cytotoxic cells that express FcγRs recognize bound antibody on a target cell and subsequently cause lysis of the target cell is referred to as antibody dependent cell-mediated cytotoxicity (ADCC) (Raghavan et al, 1996, Annu Rev Cell Dev Biol 12:181-220; Ghetie et al., 2000, Annu Rev Immunol 18:739-766; Ravetch et al., 2001, Annu Rev Immunol 19:275-290; all expressly incorporated by reference). The cell-mediated reaction wherein nonspecific cytotoxic cells that express FcγRs recognize bound antibody on a target cell and subsequently cause phagocytosis of the target cell is referred to as antibody dependent cell-mediated phagocytosis (ADCP).

There remains a need in the art for variant structures of anti-TF antibodies with properties optimized for specific clinical indications. For example, optimizing ADCC and CDC antibody functions is generally desirable for oncology indications. Other potential uses for anti-TF antibodies with enhanced ADCC activity include therapy for age related macular degeneration or other angiogenesis related conditions in which endothelial cells in aberrant blood vessels may express TF and can be targeted by ADCC.

### SUMMARY OF THE INVENTION

The present invention provides isolated anti-tissue factor antibodies, immunoglobulins, and other specified portions and variants thereof having enhanced ADCC activity, as well as anti-tissue factor antibody compositions, encoding or complementary nucleic acids, vectors, host cells, compositions, formulations, devices, transgenic animals, transgenic plants, and methods of making and using thereof, as described and enabled herein, in combination with what is known in the art. The antibodies of the invention bind human tissue factor, have modified Fc regions as compared to wild-type CNTO 860 or other Fc regions, and demonstrate enhanced ADCC activity as compared to one or more antibodies previously known in the art. Accordingly, the antibodies can be used in a variety of methods for diagnosing, treating, and/or preventing diseases involving tissue factor, where enhanced ADCC activity is desirable, such as cancer.

In one embodiment, the antibody according to the present invention includes any protein or peptide containing molecule that comprises at least a portion of a complementarity determining region (CDR) of a heavy or light chain or a ligand binding portion thereof derived from the antibody designated TF8-5G9, in combination with a heavy chain or light chain framework region, and a heavy chain or light chain constant region that is capable of interacting with receptors on effector cells or molecules to activate innate immunity (e.g., complement lysis, NK cell killing, opsonization, or phagocytosis by macrophages) and thus imparts ADCC activity to the antibody, or any portion thereof, that can be incorporated into an antibody of the present invention. The antibodies described herein are variants of a human tissue factor antibody derived from the TF8-5G9 antibody comprising a human IgG1 Fc and known as CNTO 860; they are known as CNTO 860 antibody Fc-variants.

Particular therapeutic antibodies of the invention include specified Fc-variants of human monoclonal antibody CNTO 860, and functionally equivalent antibodies which have the human heavy chain and human light chain variable amino acid sequences as set forth in SEQ ID NO: 2 (residues 1 to 117) and SEQ ID NO: 4 (residues 1 to 108), respectively, and conservative modifications thereof. The antibody amino acid sequence further comprises at least one specified substitution, insertion or deletion as described herein or as known in the art. In one embodiment, the CNTO 860 heavy chain substitution is selected from A330Y, A330I, and I332E where the I332E variant may optionally further comprise a second substitution selected from A330I, V264I, and S239D.

The invention further provides compositions comprising the CNTO860 antibody variants, such as pharmaceutical compositions further comprising pharmaceutically acceptable diluents, buffers, additives, preservatives, and stabilizers.

The invention provides methods of using the CNTO 860 antibody variants and compositions thereof to prevent or treat subjects in need thereof, particularly subjects diagnosed with or at risk of having diseases for which tissue factor activity is known to play a role in the pathology of one or more cells, tissues, or organs.

### DESCRIPTION OF THE DRAWINGS

FIG. 1 is an alignment of the amino acid sequences of the mature heavy chains of CNTO859 comprising a human IgG4 Fc domain and CNTO860 comprising a human IgG1-type Fc-domain where the differences in residues are marked in bold: the positions of substitutions in the Fc-variants of CNTO 860, extending from the hinge core motif CPPC (residue 226 Kabat numbering) to the C-terminus of the Fc domain are shown underlined.

FIG. 2 is a schematic representation of the CHO cell expression plasmids, where p4157 encoding the I332E variant is shown as an example of the heavy chain plasmids listed in Table 3. All other heavy chain plasmids had the same structure except for the mutations introduced. p4146 encodes the normal light chain for CNTO 860, which was used to express in CHO cells all variants analyzed here. In both cases, expression of the antibody sequence is driven by the CMV promoter shown.

FIG. 3 shows schematic representations of the YB2/0 cell expression plasmids: p4148 (A) encoding the I332E variant is shown as an example of the heavy chain plasmids listed in Table 3, p2402 (B) encodes the light chain for CNTO 860, which was used to express in YB2/0 cells all of the variants described herein, where expression of both the heavy and light chain sequences is driven by naturally occurring heavy and light chain promoters.

FIG. 4 shows representative data from ADCC assays using human PBMCs as the effector cells in the presence of human colorectal carcinoma cells, HCT116, and varying amounts of test Mab. The amount of specific cell lysis was determined after 2 hrs.

### DESCRIPTION OF THE SEQUENCE LISTING

| SEQ ID NO: | Description | Type | Length |
|---|---|---|---|
| 1 | CNTO860 Heavy Chain | nt | 1341 |
| 2 | CNTO860 Heavy Chain | aa | 447 |
| 3 | CNTO860 Light Chain | nt | 645 |
| 4 | CNTO860 Light Chain | aa | 214 |
| 5 | Primer I332E | nt | |
| 6 | Primer I332E | nt | |
| 7 | Primer A330I | nt | |
| 8 | Primer A330I | nt | |
| 9 | Primer A330Y | nt | |
| 10 | Primer A330Y | nt | |
| 11 | Primer I332E/A330I-QC1 | nt | |
| 12 | Primer I332E/A330I-QC2 | nt | |
| 13 | Primer I332E/V264I-QC1 | nt | |
| 14 | Primer I332E/V264I-QC2 | nt | |
| 15 | Primer I332E/S239D-QC1 | nt | |
| 16 | Primer I332E/S239D-QC2 | nt | |

### DETAILED DESCRIPTION OF THE INVENTION

### Definitions

In order that the present invention may be more readily understood, certain terms are first defined. Additional definitions are set forth throughout the detailed description.

The term “antibody” herein is used in the broadest sense and specifically covers monoclonal antibodies (including full length monoclonal antibodies and antibody variants described herein), polyclonal antibodies, multispecific antibodies (e.g., bispecific antibodies), and antibody fragments so long as they exhibit the desired biological activity and comprise an Fc-domain as defined herein.

By “CNTO860 antibody,” “CNTO 860,” or “CNTO 860 Mab” is meant a human tissue factor specific antibody wherein the binding domains including the FR1, CDR1, FR2, CDR2, FR3, CDR3, and FR4 are represented by specified regions of SEQ ID Nos: 2 and 4 for the heavy and light chain variable domains, respectively, and as disclosed in U.S. patent application Ser. No. 11/010,797. The term “CNTO860 variants” as used herein includes CNTO 860 antibodies wherein one or more amino acids of the heavy chain (SEQ ID NO: 2) have been replaced, deleted, or added as disclosed herein.

The term “ADCC activity” stands for antibody-dependent cell-mediated cytotoxicity and means the phenomenon of antibody-mediated target cell destruction by non-sensitized effector cells. The identity of the target cell varies, but it must have bound surface immunoglobulin G whose Fc portion is intact. The effector cell is a “killer” cell possessing Fc receptors. It may be a lymphocyte lacking conventional B- or T-cell markers, or a monocyte, macrophage, or polynuclear leukocyte, depending on the identity of the target cell. The reaction is complement independent. The ADCC activity of an antibody of the present invention is “enhanced” if its ability to demonstrate ADCC mediated cell killing surpasses the ability of an unmodified antibody, e.g., anti-TF IgG₁, as determined in a standard in vivo or in vitro assay of cell killing, such as the assays described herein. Preferably, the anti-TF with enhanced ADCC activity achieves the same effect (prevention or inhibition of tumor cell growth) at a lower dose and/or in a shorter time than a reference IgG1 antibody. Preferably, the difference between the potency of an antibody within the scope of the present invention and a reference antibody is at least about 1-fold, more preferably, at least about 2-fold, even more preferably, at least about 3-fold, most preferably, at least about 5-fold, as determined by side-by-side comparison in a selected standard chromium release ADCC assay.

“Effector functions” of antibodies or antibody analogs as it is used herein are processes by which pathogens or abnormal cells, e.g., tumor cells, are destroyed and removed from the body. Innate and adaptive immune responses use most of the same effector mechanisms to eliminate pathogens including ADCC, CA (complement activation), C1q binding, and opsinization.

The terms “Fc,” “Fc-containing protein” or “Fc-containing molecule” as used herein refer to a monomeric, dimeric or heterodimeric protein having at least an immunoglobulin CH₂ and CH3 domain. The CH2 and CH3 domains can form at least a part of the dimeric region of the protein molecule (e.g., antibody) when functionally linked to a dimerizing or multimerizing domain, such as the antibody hinge domain. The Fc portion of the antibody molecule (fragment crystallizable, or fragment complement binding) denotes one of the well characterized fragments produced by digestion of an antibody with various peptidases, typically papain. While various antibody fragments are defined in terms of the digestion of an intact antibody, one of skill will appreciate that such Fc fragments may be synthesized de novo either chemically or by recombinant DNA methodology, peptide display, or the like. The constant region of antibody refers to a region other than the variable region proposed by Kabat et al. (Kabat, “Sequence of Proteins of Immunological Interest,” U.S. Department of Health and Human Services (1983)). The Fc moiety refers to a region which is not involved in the binding with the antigen and which is primarily responsible for the effector function among the fragments cleaved with a proteolytic enzyme, papain. In one aspect, the Fc-containing protein of the invention is formed through the complexing (multimerizing) of Fc polypeptide sequences. By “Fc polypeptide sequences” is meant domains that typically comprise an Fc as defined above. The individual polypeptides of the dimeric structure may or may not have the same sequences and/or domains, provided they are capable of dimerizing to form an Fc region (as defined herein).

“Fc receptor” or “FcR” describes a receptor that binds to the Fc region of an antibody or fusion protein. A well-known FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see review M. in Daeron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126. 330-4 1 (1995). Other FcRs, including those to be identified in the future, are encompassed by the term “FcR” herein. The term also includes the neonatal receptor, FcRn, which is responsible for the transfer, of maternal IgGs to the fetus (Guyer et al., J. Immunol. 117:587 (1976) and Kim et al., J. Immunol. 24:249 (1994)

“Human effector cells” are leukocytes which express one or more FcRs and perform effector functions. Preferably, the cells express at least FcγRIII and perform ADCC effector function. Examples of human leukocytes which mediate ADCC include peripheral blood mononuclear cells (PBMC), natural killer (NK) cells, monocytes, cytotoxic T cells and neutrophils; with PBMCs and NK cells being preferred. The effector cells may be isolated from a native source, e.g., from blood.

“Complement dependent cytotoxicity” or “CDC” refers to the lysis of a target cell in the presence of complement. Activation of the classical complement pathway is initiated by the binding of the first component of the complement system (Clq) to antibodies (of the appropriate subclass) which are bound to their cognate antigen. To assess complement activation, a CDC assay, e.g. as described in Gazzano-Santoro et al., J. Immunol. Methods 202:163 (1996) may be performed.

The term “monoclonal antibody” as used herein is a specific form of Fc-containing protein comprising at least one ligand binding domain which retains substantial homology to at least one of a heavy or light chain antibody variable domain of at least one species of animal antibody and which binding domains have a specific and defined affinity for an antigen or epitope of that antigen. The terms “monoclonal antibody” or “monoclonal antibody composition” as used herein refer to a preparation of antibody molecules of single molecular composition. A monoclonal antibody composition displays a single binding specificity and affinity for a particular epitope. Further, a monoclonal antibody, as used herein, is intended to refer to an antibody which is isolated and therefore substantially free of other antibodies having different antigenic specificities, e.g., an antibody which is capable of being isolated on the basis of its known composition or specificity.

The terms “tissue factor protein,” “TF,” and “mammalian tissue factor protein” are used to refer to a polypeptide having an amino acid sequence corresponding to a naturally occurring mammalian tissue factor or a recombinant tissue factor also known as coagulation factor III and CD142. Naturally occurring TF includes human species (NCBI Accession No. NP_{—}001984) as well as other animal species, such as rabbit, rat, porcine, non human primate, equine, murine, and ovine tissue factor. The amino acid sequence of the other mammalian tissue factor proteins are generally known or obtainable through conventional techniques.

A “TF mediated or associated process or activity,” or equivalently, or “TF activity,” according to the present invention is any biological activity which is mediated by the presence of TF. A “TF related disease or disorder” is meant to diseases or disorders which may be impacted through the inhibition of TF, particularly the inhibition of tumor growth on tissue factor expressing cells, but also includes other tissue factor mediated diseases and processes.

The term “epitope” means a protein determinant capable of specific binding to an antibody. Epitopes usually consist of chemically active surface groupings of molecules, such as amino acids or sugar side chains, and usually have specific three-dimensional structural characteristics, as well as specific charge characteristics. Conformational and nonconformational epitopes are distinguished in that the binding to the former but not the latter is lost in the presence of denaturing solvents. The term “native conformational epitope” or “native protein epitope” are used interchangeably herein, and include protein epitopes resulting from conformational folding of the integrin molecule which arise when amino acids from differing portions of the linear sequence of the integrin molecule come together in close proximity in 3-dimensional space. Such conformational epitopes are distributed on the extracellular side of the plasma membrane.

As used herein, “specific binding” refers to antibody binding to a predetermined antigen. Typically, the antibody binds with a dissociation constant (K_{D}) of 10⁻⁷ M or less, and binds to the predetermined antigen with a K_{D} that is at least twofold less than its K_{D} afor binding to a non-specific antigen (e.g., BSA, casein) other than the predetermined antigen or a closely-related antigen. The phrases “an antibody recognizing” an antigen and “an antibody specific for” an antigen, e.g., TF, are used interchangeably herein with the term “an antibody which binds specifically to” an antigen. An antibody, due to its dimerized or multimerized structure is typically divalent or multivalent for antigen binding. It will be appreciated that, through standard techniques known in the art, antigen binding domains may be exchanged to from bispecific or multispecific antibodies.

As used herein, the term “high affinity” for an IgG antibody refers to an antibody having a K_{D} Of 10⁻⁸ M or less, more preferably 10⁻⁹ M or less and even more preferably 10⁻¹⁰ M or less. However, “high affinity” binding can vary for other antibody isotypes. For example, “high affinity” binding for an IgM isotype refers to an antibody having a K_{D} of 10⁻⁷ M or less, more preferably 10⁻⁸ M or less. The term “K_{assoc}” or “Kₐ,” as used herein, is intended to refer to the association rate of a particular antibody-antigen interaction, whereas the term “K_{dis}” or “K_{d},” as used herein, is intended to refer to the dissociation rate of a particular antibody-antigen interaction, The term “K_{D}”, as used herein, is intended to refer to the dissociation constant, which is obtained from the ratio of K_{d} to Kₐ (i.e., K_{d}/Kₐ) and is expressed as a molar concentration (M). K_{D} may also be derived or determined from measured “on” (kₒₙ) and “off” (k_{off}) rates of antibody association with and antigen, where is K_{D} is kₒₙ/k_{off}.

As used herein, antibody “isotype” or “class” refers to the IgA, IgD, IgE, IgG, or IgM designation that is encoded by heavy chain constant region genes. Among human IgG isotypes there are four subclasses; IgG1, IgG2, IgG3 and IgG4 named in order of their natural abundance in serum starting from highest to lowest. IgA antibodies are found as two subclasses, IgA1 and IgA2. As used herein, “isotype switching” also refers to a change between IgG subclasses or subtypes.

The term “nucleic acid molecule,” as used herein, is intended to include DNA molecules and RNA molecules. A nucleic acid molecule may be single-stranded or double-stranded, but preferably is double-stranded DNA. A nucleic acid molecule, as used herein in reference to nucleic acids encoding antibodies or antibody portions (e.g., VH, VL, CDR3) that bind to tissue factor, is intended to refer to a nucleic acid molecule in which the nucleotide sequences encoding the antibody or antibody portion are capable of being isolated and free of other nucleotide sequences encoding antibodies or antibody portions that bind antigens other than tissue factor. In one embodiment, the anti-tissue factor antibody, or portion thereof, includes the isolated nucleotide or amino acid sequence of a CNTO 860 antibody variant.

As used herein, the term “subject” includes any human or nonhuman animal. The term “nonhuman animal” includes all vertebrates, e.g., mammals and nonmammals, such as nonhuman primates, sheep, dog, cow, chickens, amphibians, reptiles, etc.

### Citations

All publications or patents cited herein are entirely incorporated herein by reference as they show the state of the art at the time of the present invention and/or to provide description and enablement of the present invention.

Publications refer to any scientific or patent publications, or any other information available in any media format, including all recorded, electronic or printed formats. The following references are entirely incorporated herein by reference: Crotty, David (Exec Ed.) Cold Spring Harbor Protocols, 2007. Cold Spring Harbor Laboratory Press. Online ISSN: 1559-6095 searchable by topic; Ausubel, et al., (Eds), Current Protocols in Molecular Biology, John Wiley & Sons, Inc., NY, N.Y. (1987-2007); Coligan, et al., (Eds), Current Protocols in Immunology, John Wiley & Sons, Inc., NY (1994-2007); Coligan et al., (Eds) Current Protocols in Protein Science, John Wiley & Sons, NY, N.Y., (1997-2007); Enna et al., (Eds), Current Protocols in Pharmacology, John Wiley & Sons, Inc., NY (1994-2006); and Wang, et al. (Eds), Drug Delivery, John Wiley & Sons, Inc., 2005, especially Ch 10-19.

Throughout the present specification and claims, the numbering of the residues in an immunoglobulin heavy chain is that of the EU index as in Kabat et al., Sequences of Proteins of Immunological Interest, 5th Ed. Public Health Service, National Institutes of Health, Bethesda, Md. (1991), expressly incorporated herein by reference. The “EU index as in Kabat” refers to the residue numbering of the human IgG1 EU antibody.

### 1. Generation, Screening, and Production of Antibodies

The present invention provides isolated, recombinant and/or synthetic anti-tissue factor monoclonal antibodies having enhanced ADCC activity, as well as compositions and encoding nucleic acid molecules comprising at least one polynucleotide encoding such antibodies.

A growing number of Abs being developed and intended for use as therapeutic agents, the inherent specificity of the antibody for its target and, additionally, the nonantigen binding functions of the antibody, such as Ab-dependent cell cytotoxicity (ADCC), to affect their therapeutic activity. The nonantigen binding functions of Mabs involve binding to Fc-receptors on immune cells and reside in the structure formed by the constant domains of the heavy chains (the Fc-domain).

The impact of the nonantigen binding activity of a therapeutic Mab on clinical outcome was recognized in Non-Hodgkin's Lymphoma patients treated with anti-CD20 Ab Rituxan having variant FcγIIIa with higher than normal IgG Fc affinity (Cartron et al., 2002, Blood 98:754). As a result, attention has focused on means to control and optimize the nonantigen binding functions of therapeutic Mab candidates. Possible advantages include: better clinical responses; a greater number of patients responding; or lower dose levels required to achieve the same degree of response, possibly reducing side effects and costs.

The strategy of identifying variants with enhanced affinity for Fc-receptors, particularly those designated Fcγ-Receptors (FcγR) seems a logical approach for enhancing ADCC activity. However, due to the number and diverse functions of FcγR types, the task of optimizing receptor binding profile to enhance therapeutic activity is complex. A therapeutically advantageous Fc-receptor binding profile may be one of optimized differential binding to the Fc-receptors as demonstrated in a murine tumor model system (Nimmerjahn and Ravetch, 2005, Science 310:1510). Table 1 provides a listing of the major classes of human and mouse FcγRs known in the present art, and shows the important classification into activating and inhibiting receptors. Receptors in the same row are considered functional orthologs of each other. It will be appreciated that signaling through inhibiting receptors at the same time as activating receptors on the same cell may block the signaling cascade that originated from the activating receptors.

TABLE 1
Fcγ Receptors in Humans and Mice

| Human | Murine |
|---|---|
| Activating FcγR | Activating FcγR |
| FcγRI | FcγRI |
| FcγRIIa | FcγRIII |
| FcγRIIIa | FcγRIV |
| Inhibiting FcγR | Inhibiting FcγR |
| FcγRIIb | FcγRII |

Applicants have prepared variants of the anti-tissue factor antibody known as CNTO 860, the wild-type antibody comprised of heavy and light chain polypeptides having the mature sequences of SEQ ID NOs: 2 and 4, respectively, and evaluated the variants for binding to Fc-receptor species as well as testing biological activity, in vitro ADCC activity, in order to identify methods of antibody engineering which produce therapeutic antibody candidates with properties of advantageous nonantigen binding functional properties. In particular, the CNTO 860 variants are considered to have advantageous nonantigen binding properties if the variant produces enhanced ADCC activity, that is target tumor cell killing activity, as compared to the unaltered (parent or wildtype) antibody.

Anti-tissue factor antibodies of the present invention can be generated by a variety of techniques, including conventional monoclonal antibody techniques, e.g., the standard hybridoma technique of Kohler and Milstein (1975) Nature 256:495. Preparation of immunogenic antigens, such as isolated tissue factor protein or a portion thereof (including synthetic molecules, such as synthetic peptides), and monoclonal antibody generation, selection, isolation, and cloning can be performed using any suitable technique.

For the production of monoclonal antibodies of the invention, a variety of cell lines, mixed cell lines, an immortalized cell or clonal population of immortalized cells, can be used, as well known in the art. In one approach, a hybridoma is produced by fusing a suitable immortal cell line (e.g., a myeloma cell line) such as, but not limited to Chinese hamster ovary (CHO)-derived cell lines, NSO and cell lines derived there from, NSO, NS1, NS2, Sp2/0 and cell lines derived therefrom, Sp2 SA3, Sp2 MAI, Sp2 SS1, Sp2 SA5, AE-1, L.5, P3X63Ag8.653, U937, MLA 144, ACT IV, MOLT4, DA-1, JURKAT, WEHI, K-562, BHK, HEK-293, COS, RAJI, NIH 3T3, HL-60, MLA 144, NAMAIWA, NEURO 2A, human retina-derived PerC.6, Y3-Ag 1.2.3 and derivatives, YB2/O or the like, or heteromyelomas, fusion products thereof, or any cell or fusion cell derived therefrom, or any other suitable cell line as known in the art (see, e.g., www.atcc.org) and the like, may be used as a fusion partner, with antibody producing cells, such as, but not limited to, isolated or cloned spleen, peripheral blood, lymph, tonsil, or other immune or B cell containing cells, or any other cells expressing heavy or light chain constant or variable or framework or CDR sequences, either as an endogenous or heterologous nucleic acid. Antibody producing cells can also be obtained from the peripheral blood or, preferably the spleen or lymph nodes, of humans or other suitable animals that have been immunized with the antigen of interest.

Preceding or following production of hybridomas, transfectomas, derivatives or clones thereof, the antibody expressed by the cell lines may be tested for specificity and affinity of binding. Both specificity and affinity (strength) of binding may be tested in liquid or solid phase formats, such as by ELISA. Screening antibodies for specific binding to similar proteins or fragments can also be conveniently achieved using peptide display libraries. Libraries of peptides may be generated either by chemical synthesis and recombinant methods, especially by using bacteriophage display methods.

Any suitable host cell can also be used for expressing heterologous or endogenous nucleic acid encoding an antibody, specified fragment or variant thereof, of the present invention selected from viral, bacterial, algal, prokaryotic, plant, amphibian, insect, reptilian, fish, mammalian, rodent, equine, ovine, goat, sheep, primate, or human. In addition to the mammalian cells listed above, especially CHO and NSO derived host cell lines; engineered Escherichia coli, Pichia pastoris, or Drosophilia melanogaster cells; transgenic plants, such as tobacco, maize, soy, rice, or wheat; and transgenic animals, such as goats or mice, may be used to produce quantities of antibodies in amounts for testing or commercial sales. Preferred mammalian host cells for expressing the recombinant antibodies of the invention include Chinese Hamster Ovary (CHO cells) (including dhfrCHO cells, described in Urlaub and Chasin, (1980) Proc. Natl. Acad. Sci. USA 77:42164220, used with a DHFR selectable marker, e.g., as described in R. J. Kaufman and P. A. Sharp (1982) Mol. Biol. 159:601-621), NSO myeloma cells, COS cells and SP2 cells. In particular, for use with NSO myeloma cells, another preferred expression system is the GS gene expression system disclosed in WO 87/04462, WO 89/01036 and EP 338,841. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods. Human antibodies of the invention also can be produced in a host cell transfectoma using, for example, a combination of recombinant DNA techniques and gene transfection methods as is well known in the art (e.g., Morrison, S. (1985) Science 229:1202).

For example, to express the antibodies, or antibody fragments thereof, DNAs encoding partial or full-length light and heavy chains, can be obtained by standard molecular biology techniques (e.g., PCR amplification, site directed mutagenesis) and can be inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). The light and heavy chain variable regions of the antibodies described herein can be used to create full-length antibody genes of any antibody isotype by inserting them into expression vectors already encoding heavy chain constant and light chain constant regions of the desired isotype such that the VH segment is operatively linked to the CH segment(s) within the vector and the VI, segment is operatively linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of the invention in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody. Prokaryotic expression of antibody genes has been reported to be ineffective for production of high yields of active antibody (Boss, M. A. and Wood, C. R. (1985) Immunology Today 6:12-13).

Antibodies of the present invention can also be prepared using at least one tissue factor antibody encoding nucleic acid to provide transgenic animals or mammals, such as goats, cows, horses, sheep, and the like, that produce such antibodies in their milk. Such animals can be provided using known methods. See, e.g., but not limited to, U.S. Pat. Nos. 5,827,690; 5,849,992; 4,873,316; 5,849,992; 5,994,616; 5,565,362; 5,304,489, and the like, each of which is entirely incorporated herein by reference.

### Host Cells

A number of suitable host cell lines capable of expressing intact glycosylated proteins have been developed in the art, and include the COS-1 (e.g., ATCC CRL 1650), COS-7 (e.g., ATCC CRL-1651), HEK293, BHK21 (e.g., ATCC CRL-10), CHO (e.g., ATCC CRL 1610) and BSC-1 (e.g., ATCC CRL-26) cell lines, Cos-7 cells, PerC.6 cells, hep G2 cells, P3X63Ag8.653, SP2/0-Ag14, 293 cells, HeLa cells and the like, which are readily available from, for example, American Type Culture Collection, Manassas, Va. (www.atcc.org). Preferred host cells include cells of lymphoid origin such as myeloma and lymphoma cells.

Antibodies of the present invention can additionally be prepared using at least one CNTO860 antibody variant encoding nucleic acid to provide transgenic plants and cultured plant cells (e.g., but not limited to tobacco, maize, rapeseed, and duckweed) that produce such antibodies, specified portions or variants in the plant parts or in cells cultured therefrom. As a non-limiting example, transgenic tobacco leaves expressing recombinant proteins have been successfully used to provide large amounts of recombinant proteins, e.g., using an inducible promoter. See, e.g., Cramer et al., Curr. Top. Microbol. Immunol. 240:95-118 (1999) and references cited therein. Also, transgenic maize have been used to express mammalian proteins at commercial production levels, with biological activities equivalent to those produced in other recombinant systems or purified from natural sources. See, e.g., Hood et al., Adv. Exp. Med. Biol. 464:127-147 (1999) and references cited therein. Antibodies have also been produced in large amounts from transgenic plant seeds including antibody fragments, such as single chain antibodies (scFv's), including tobacco seeds and potato tubers. See, e.g., Conrad et al., Plant Mol. Biol. 38:101-109 (1998) and reference cited therein. Thus, antibodies of the present invention can also be produced using transgenic plants, according to know methods. See also, e.g., Fischer et al., Biotechnol. Appl. Biochem. 30:99-108 (October, 1999), Ma et al., Trends Biotechnol. 13:522-7 (1995); Ma et al., Plant Physiol. 109:341-6 (1995); Whitelam et al., Biochem. Soc. Trans. 22:940-944 (1994); and references cited therein. Each of the above references is entirely incorporated herein by reference.

As disclosed and claimed herein, the sequences set forth in SEQ ID NOs. 2 and 4 include “conservative sequence modifications,” i.e., amino acid sequence modifications which do not significantly affect or alter the binding characteristics of the antibody encoded by the nucleotide sequence or containing the amino acid sequence. Such conservative sequence modifications include amino acid substitutions, additions and deletions. Conservative amino acid substitutions include ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, a predicted nonessential amino acid residue in a tissue factor antibody is preferably replaced with another amino acid residue from the same side chain family.

### 2. Nucleic Acid Molecules and Production Cell Lines

Using the information provided herein, such as the nucleotide sequences encoding at least 70-100% of the contiguous amino acids of SEQ ID NOS: 1, specified fragments, variants or consensus sequences thereof, or a vector comprising at least one of these sequences, a nucleic acid molecule of the present invention encoding at least one anti-tissue factor antibody which is a CNTO 860 antibody Fc-variant can be obtained using methods described herein or as known in the art.

Isolated nucleic acid molecules of the present invention can include nucleic acid molecules comprising the coding sequence for, but not limited to, at least one specified portion of at least one CDR, as CDR1, CDR2 and/or CDR3 of at least one heavy chain of SEQ ID NO: 1 or of light chain SEQ ID NO: 3; and nucleic acid molecules which comprise a nucleotide sequence substantially different from those described above but which, due to the degeneracy of the genetic code, still encode at least one anti-tissue factor antibody as described herein and/or as known in the art. Of course, the genetic code is well known in the art. Thus, it would be routine for one skilled in the art to generate such degenerate nucleic acid variants that code for specific anti-tissue factor antibodies of the present invention. See, e.g., Ausubel, et al., supra, and such nucleic acid variants are included in the present invention.

Modifications can be introduced into SEQ ID NOs: 1 and 3 by standard techniques known in the art, such as site-directed mutagenesis and PCR-mediated mutagenesis. Codon substitutions in SEQ ID NOs: 1 and 3 which do not alter the sequence of the encoded protein are also included in the present invention. Codon substitutions of the coding sequence are often desirable when the expression system for the antibody is altered, e.g., from a murine myeloma cell line to an E. coli system. Alternatively, in another embodiment, mutations can be introduced randomly along all or part of a anti-tissue factor antibody coding sequence, such as by saturation mutagenesis, and the resulting modified anti-tissue factor antibodies can be screened for binding activity.

Accordingly, antibodies encoded by the nucleotide sequences disclosed herein and/or containing the amino acid sequences disclosed herein (i.e., SEQ ID NOs: 2 and 4) include substantially similar antibodies encoded by or containing similar sequences which have been conservatively modified. The percent identity between two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package (available at www.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences can also determined using the algorithm of E. Meyers and W. Miller (Comput. Appl. Biosci., 4:11-17 (1988)) which has been incorporated into the ALIGN program (version 2.0), using a PAM 1 20 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (www.gcg.com), using either a Blossum 62 matrix or a PAM2 5 0 matrix, and a gap weight of 16, 14, 12, 10, 8, 6, or 4 and a length weight of 1, 2, 3, 4, 5, or 6. The nucleic acid and protein sequences of the present invention can further be used as a “query sequence” to perform a search against public databases to, for example, identify related sequences. Such searches can be performed using the NBLAST and XBLAST programs (version 2.0) of Altschul, et al. (1990) J. Mol. Biol. 215.403-1 0. BLAST nucleotide searches can be performed with the NBLAST program, score=100, wordlength=12 to obtain nucleotide sequences homologous to the nucleic acid molecules of the invention. BLAST protein searches can be performed with the XBLAST program, score=50, wordlength=3 to obtain amino acid sequences homologous to the protein molecules of the invention. To obtain gapped alignments for comparison purposes, Gapped BLAST can be utilized as described in Altschul et al., (1997) Nucleic Acids Res. 25(17):3389. When utilizing BLAST and Gapped BLAST programs, the default parameters of the respective programs (e.g., XBLAST and NBLAST) can be used. See http://www. ncbi.nlm.nih.gov.

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The term “recombinant host cell” (or simply “host cell”), as used herein, is intended to refer to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. Recombinant host cells include, for example, CHO cells and lymphocytic cells.

In another aspect, the invention provides isolated nucleic acid molecules encoding a(n) anti-tissue factor, CNTO 860 antibody variants, having an amino acid sequence as encoded by the nucleic acid contained in the plasmid designated clone p2401.

As indicated herein, nucleic acid molecules of the present invention which comprise a nucleic acid encoding a CNTO 860 antibody Fc-variant can include additional coding sequence that codes for additional amino acids, such as those that provide additional functionalities. Thus, the sequence encoding an antibody can be fused to a marker sequence, such as a sequence encoding a peptide that facilitates purification of the fused antibody comprising an antibody fragment or portion. Alternatively, the CNTO 860 antibody Fc-variant of the invention may be fused to another polypeptide which imparts additional biological or therapeutic activity to the antibody such as a cytokine moiety or a second binding domain. Such fusion constructs are known and methods of making them have been described in the art.

The present invention provides isolated nucleic acids that hybridize under selective hybridization conditions to a polynucleotide disclosed herein. Thus, the polynucleotides of this embodiment can be used for isolating, detecting, modifying, or quantifying nucleic acids comprising such polynucleotides. Exemplary nucleic acids include SEQ ID Nos: 5-16. For example, polynucleotides of the present invention can be used to identify, isolate, or amplify partial or full-length clones in a deposited library. In some embodiments, the polynucleotides are genomic or cDNA sequences isolated, or otherwise complementary to, a cDNA from a human or mammalian nucleic acid library.

The isolated nucleic acids of the present invention can be made using (a) recombinant methods, (b) synthetic techniques, (c) purification techniques, or combinations thereof, as well-known in the art. Additional sequences can be added to such cloning and/or expression sequences to optimize their function in cloning and/or expression, to aid in isolation of the polynucleotide, or to improve the introduction of the polynucleotide into a cell. Use of cloning vectors, expression vectors, adapters, and linkers is well known in the art. (See, e.g., Ausubel, supra; or Crotty, supra). Chemical synthesis generally produces a single-stranded oligonucleotide, which can be converted into double-stranded DNA by hybridization with a complementary sequence, or by polymerization with a DNA polymerase using the single strand as a template. One of skill in the art will recognize that while chemical synthesis of DNA can be limited to sequences of about 100 or more bases, longer sequences can be obtained by the ligation of shorter sequences. Such a method of constructing functional dsDNA molecules is taught in U.S. Pat. No. 6,521,427 and WO02081490.

As described herein, the present invention further provides recombinant expression plasmids comprising a nucleic acid of the present invention. A recombinant expression plasmid or cassette will typically comprise a polynucleotide of the present invention operably linked to transcriptional initiation regulatory sequences that will direct the transcription of the polynucleotide in the intended host cell. Both heterologous and non-heterologous (i.e., endogenous) promoters can be employed to direct expression of the nucleic acids of the present invention.

In addition to the antibody chain genes, the recombinant expression vectors of the invention carry regulatory sequences that control the expression of the antibody chain genes in a host cell. The term “regulatory sequence” is intended to includes promoters, enhancers and other expression control elements (e.g., polyadenylation signals) that control the transcription or translation of the antibody chain genes. Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP)) and polyoma. Alternatively, nonviral regulatory sequences may be used, such as the ubiquitin promoter or P-globin promoter. Expression vectors for these cells can include one or more of the following expression control sequences, such as, but not limited to an origin of replication; a promoter (e.g., late or early SV40 promoters, the CMV promoter (U.S. Pat. Nos. 5,168,062; 5,385,839), an HSV tk promoter, a pgk (phosphoglycerate kinase) promoter, an EF-1 alpha promoter (U.S. Pat. No. 5,266,491), at least one human immunoglobulin promoter; an enhancer, and/or processing information sites, such as ribosome binding sites, RNA splice sites, polyadenylation sites (e.g., an SV40 large T Ag poly A addition site), and transcriptional terminator sequences.

Various cis-acting DNA elements have been incorporated into vectors. For vector engineering, such DNA elements should have relatively small size (>6 kb), universal function and, desirably, the ability to confer copy number dependence (such that expression is directly correlated to the number of copies of vector incorporated into the genome, which has relevant advantages in amplification procedures). Elements, such as locus control regions and insulators, are among these. A wide range of other elements: antirepressor or STAR (stabilising and antirepressor) elements, which are used to flank transgenes in mammalian expression vectors, affect the spread of methylation and histone deacetylation patterns from the surrounding genome into the recombinant DNA and scaffold/matrix-associated regions (S/MARs), which bind to the nuclear matrix, are among these. Ubiquitous chromatin opening elements (UCOEs) are elements derived from the promoters of housekeeping genes. Housekeeping genes are usually transcriptionally active owing to a significant extent of histone acetylation and the inclusion of UCOEs in expression vectors can increase production and stability of transgene expression in CHO cells. Others have reported that the flanking of transgenes with 5′ and 3′ sequences from highly expressed housekeeping genes, such as the elongation factor-1α gene, can lead to significantly increased production from transgenes in a range of mammalian cell lines. See Barnes and Dickson, 2006. Current Opinion Biotechnol. 17(4): 381-386, for a review.

As an alternative to the inclusion of regulatory elements within expression vectors, a second approach has focused on alteration of the general epigenetic environment of the chromatin surrounding the sites of transgene insertion. Histone acetylation, which is generally associated with enhanced transcription, arises from the balance of histone acetyltransferase (HAT) and histone deacetylase (HDAC) activities.

In addition to the antibody chain genes and regulatory sequences, the recombinant expression vectors of the invention may carry additional selectable marker genes. The selectable marker gene facilitates selection of host cells into which the vector has been introduced (see, e.g., U.S. Pat. Nos. 4,399,216; 4,634,665 and 5,179,017, all by Axel et al.). Selectable marker genes include the dihydrofolate reductase (DHFR) gene (for use in dhfr-host cells with methotrexate selection/amplification) and the neo gene (for G418 selection). Expression vectors will preferably but optionally include at least one selectable marker. Such markers include, e.g., but not limited to, methotrexate (MTX), dihydrofolate reductase (DHFR, U.S. Pat. Nos. 4,399,216; 4,634,665; 4,656,134; 4,956,288; 5,149,636; 5,179,017, ampicillin, neomycin (G418), mycophenolic acid, or glutamine synthetase (GS, U.S. Pat. Nos. 5,122,464; 5,770,359; 5,827,739) resistance for eukaryotic cell culture, and tetracycline or ampicillin resistance genes for culturing in E. coli and other bacteria or prokaryotics (the above patents are entirely incorporated hereby by reference). Appropriate culture mediums and conditions for the above-described host cells are known in the art.

The use of selectable markers allows for amplification of the recombinant gene number and can lead to enhanced transcriptional efficiency from transgenes. Alternatively, cloning for productive cell lines after each amplification step may result in more productive clones than the repeated amplification of cell line pools and cloning at the final stage of amplification. Thus, the CNTO860 antibodies of the invention may be produced in cell lines which are selected derivative clones of cell lines into which the nucleic acid sequences coding for the antibody have been introduced.

At least one antibody of the present invention can be expressed in a modified form, such as a fusion protein, and can include not only secretion signals, but also additional heterologous functional regions. For instance, a region of additional amino acids, particularly charged amino acids, can be added to the N-terminus of an antibody to improve stability and persistence in the host cell, during purification, or during subsequent handling and storage. Also, peptide moieties can be added to an antibody of the present invention to facilitate purification. Such regions can be removed prior to final preparation of an antibody or at least one fragment thereof. Such methods are described in many standard laboratory manuals, such as Crotty, supra, see e.g. Tagging Proteins or Cloning by PCR; Ausubel, supra, Chapters 16, 17 and 18.

Those of ordinary skill in the art are knowledgeable in the numerous expression systems available for expression of a nucleic acid encoding a protein of the present invention. Further, there are numerous host cell lines suitable as recipients of the nucleic acids coding for the antibody polypeptides of the invention within vectors and operably linked to nucleic acid sequences which promote, enhance, direct, regulate or otherwise cause the expression of the encoded antibody sequences.

### 3. Purification of the Antibody

A CNTO860 antibody variant of the invention can be recovered and purified from recombinant cell cultures by well-known methods which typically involve filtration steps followed by chromatography on various types of materials including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be employed for purification. See, e.g., Coligan, Current Protocols in Immunology, or Current Protocols in Protein Science, John Wiley & Sons, NY, N.Y., (2007), e.g., Chapters 1, 4, 6, 8, 9, 10, each entirely incorporated herein by reference.

Antibodies of the present invention include products of chemical synthetic procedures, and products produced by recombinant techniques from a eukaryotic host, including, for example, yeast, higher plant, insect and mammalian cells. Depending upon the host employed in a recombinant production procedure, the antibody of the present invention can be glycosylated or can be non-glycosylated, with glycosylated preferred. Such methods are described in many standard laboratory manuals, such as Ausubel, supra, Chapters 10, 12, 13, 16, 18 and 20, Coligan, Protein Science, supra, Chapters 12-14, all entirely incorporated herein by reference.

### 4. Anti-Tissue Factor Antibodies of the Invention

Since it is well known in the art that antibody heavy and light chain CDR domains impart the binding specificity/affinity of an antibody for an antigen, the recombinant antibodies of the invention prepared as set forth above preferably comprise the heavy and light chain CDRs of CNTO 860 noted as specific residues within the sequences of the heavy and light chain variable regions of SEQ ID Nos: 2 and 4, respectively. The non-CDR regions within the variable domains of the heavy and light chain framework regions comprise what are known as the framework regions (FR1, FR2, FR3, and FR4) where a complete variable domain is comprised of FR1-CDR1-FR2-CDR2-FR3-CDR3-FR4). In a preferred embodiment the three heavy chain CDRs and the three light chain CDRs of the antibody or antigen-binding fragment have the amino acid sequence of the corresponding CDR of CNTO 860, as described herein. Such antibodies can be prepared by chemically joining together the various portions (e.g., CDRs and framework portions, FR1, FR2, FR3, and FR4) of the antibody using conventional techniques, by preparing and expressing a (i.e., one or more) nucleic acid molecule that encodes the antibody using conventional techniques of recombinant DNA technology or by using any other suitable method.

Preferably, the CDR1, 2, and/or 3 of the engineered antibodies described above comprise the exact amino acid sequence(s) as those of CNTO 860 disclosed herein. However, the ordinarily skilled artisan will appreciate that some deviation from the exact CDR sequences of CNTO 860 may be possible while still retaining the ability of the antibody to bind human tissue factor effectively (e.g., conservative substitutions). Accordingly, in another embodiment, the engineered antibody may be composed of one or more CDRs that are, for example, 90%, 95%, 98% or 99.5% identical to one or more CDRs of CNTO 860.

In addition to binding tissue factor, the CNTO 860 antibody variants of the invention bind an Fc-receptor, such as FcγRI, FcγRII, FcγRIII, FcγRIV.

Engineered antibodies such as those described above may be selected for their retention of other functional properties of antibodies of the invention, such as:
1) binding to live cells expressing human tissue factor;
2) binding to human tissue factor with a K_{D} of 10⁻⁸ M or less (e.g., 10⁻⁹ M or 10⁻¹⁰ M or less);
3) binding to the unique epitope on tissue factor recognized by the TF8-5G9 antibody;
4) inhibition of the growth of tumor cells in vivo; and
5) binding to an Fc-receptor on the surface of an immune effector cell.

Examplary methods for determining if the selected human anti-tissue factor monoclonal antibodies bind to unique epitopes, include labeling the antibody to be tested by biotinylation using commercially available reagents (Pierce, Rockford, Ill.). Competition studies using unlabeled monoclonal antibodies and biotinylated monoclonal antibodies can be performed using tissue factor coated-ELISA plates. Biotinylated mAb binding can be detected with a streptavidin-alkaline phosphatase probe. To determine the isotype of purified antibodies, isotype ELISAs can be performed. In order to demonstrate binding of monoclonal antibodies to live cells expressing the tissue factor, flow cytometry can be used. Anti-tissue factor human IgGs can be further tested for reactivity with tissue factor antigen by Western blotting.

An antibody of the invention can be of any class (IgG, IgA, IgM, IgE, IgD, etc.) or that contains an Fc receptor binding domain and thus has the desired spectrum of effector functions conferred by that isotype and subclass and can comprise a kappa or lambda light chain. Quantifiable properties of antibody isotypes and subclasses thought to confer in vivo activities such as ADCC, CA, and opsinization are shown below (Table 2) and described in e.g. Janeway et al. eds., 2001. Immunobiology: The immune system in health an disease, Garland Publishing, NY, N.Y., USA, Chapters 4 and 9. In one embodiment, the antibody comprises an IgG heavy chain or defined fragment, for example, at least one of isotypes, IgG1, IgG2, IgG3 or IgG4, preferably an IgG1 class. In another embodiment, the anti-human tissue factor human antibody comprises an IgG1 heavy chain and an IgGkapp, light chain.

TABLE 2

| | IgG1 | IgG2 | IgG3 | IgG4 | IgM | IgA | IgE | IgD |
|---|---|---|---|---|---|---|---|---|
| Complement | ++ | + | +++ | − | +++ | + | − | − |
| Activation | | | | | | | | |
| Phagocyte | + | − | + | +/− | − | − | − | − |
| Binding | | | | | | | | |
| Neutralization | ++ | ++ | ++ | ++ | ++ | + | − | − |
| Opsinization | +++ | +/− | ++ | + | − | + | − | − |
| Sensitization | ++ | − | ++ | − | − | − | − | − |
| for killing by NKs | | | | | | | | |
| Sensitization | + | − | + | − | − | − | − | +++ |
| of Mast cells | | | | | | | | |
| Extravascular | +++ | +++ | +++ | +++ | +/− | ++ sIgA | − | + |
| diffusion | | | | | | | | |

The different IgG subclasses have different affinities for the FcγRs, with IgG1 and IgG3 typically binding substantially better to the receptors than IgG2 and IgG4 (Jefferis et al., 2002, Immunol Lett 82:57-65). All FcγRs bind the same region on IgG Fc, yet with different affinities: the high affinity binder FcγRI has a Kd for IgG1 of 10^{8 M-1}, whereas the low affinity receptors FcγRII and FcγRIII generally bind at 10⁶ and io respectively. The extracellular domains of FcγRIIIa and FcγRIIIb are 96% identical, however FcγRIIIb does not have an intracellular signaling domain. As noted above, whereas FcγRI, FcγRIIa/c, and FcγRIIIa are positive regulators of immune complex-triggered activation, FcγRIIb is inhibitory. Thus, the former are referred to as activation receptors, and FcγRIIb is referred to as an inhibitory receptor. The receptors also differ in expression pattern and levels on different immune cells. Yet another level of complexity is the existence of a number of FcγR polymorphisms in the human proteome. A particularly relevant polymorphism with clinical significance is VI 58/F1 58 FcγRIIIa. Human IgGI binds with greater affinity to the VI 58 allotype than to the F1 58 allotype. This difference in affinity, and presumably its effect on ADCC and/or ADCP, has been shown to be a significant determinant of the efficacy of the anti-CD2O antibody rituximab (Rituxan®, a registered trademark of IDEC Pharmaceuticals Corporation). Patients with the VI 58 allotype respond favorably to rituximab treatment; however, patients with the lower affinity F1 58 allotype respond poorly (Cartron et al, 2002, Blood 99:754-758, expressly incorporated by reference). Approximately 10-20% of humans are VI 58N1 58 homozygous, 45% are VI 58/F1 58 heterozygous, and 35-45% of humans are F1 58/F1 58 homozygous (Lehrnbecher et al., 1999, Blood 94:4220-4232; Cartron et al., 2002, supra). Thus, 80-90% of humans are poor responders, that is they have at least one allele of the F158 FcγRIIIa.

An overlapping but separate site on Fc, serves as the interface for the complement protein CIq. In the same way that Fc/FcγR binding mediates ADCC, Fc/CIq binding mediates complement dependent cytotoxicity (CDC). A site on Fc between the CH2 and CH3 domains, mediates interaction with the neonatal receptor, FcRn, the binding of which recycles endocytosed antibody from the endosome back to the bloodstream (Raghavan et al., 1996, Annu Rev Cell Dev Biol 12:181-220; Ghetie et al., 2000, Annu Rev Immunol 18:739-766). This process, coupled with preclusion of kidney filtration due to the large size of the full length molecule, results in favorable antibody serum half-lives ranging from one to three weeks. Binding of Fc to FcRn also plays a key role in antibody transport. The binding site for FcRn on Fc is also the site at which the bacterial proteins A and G bind. The tight binding by these proteins is typically exploited as a means to purify antibodies by employing protein A or protein G affinity chromatography during protein purification.

In another aspect of the invention, the structural features of a human anti-tissue factor antibody of the invention, CNTO 860, are used to create structurally related human anti-tissue factor antibodies that retain the functional properties of the antibodies of the invention, i.e., the binding to human tissue factor and binding to an Fc receptor.

The antibodies of the invention can bind human tissue factor with a wide range of affinities (K_{D}). In a preferred embodiment, at least one human mAb of the present invention can optionally bind human tissue factor with high affinity. For example, a human mAb can bind human tissue factor with a K_{D} equal to or less than about 10⁻⁷ M, such as but not limited to, 0.1-9.9 (or any range or value therein) X 10⁻⁷, 10⁻⁸, 10⁻⁹, 10⁻¹⁰, 10¹¹, 10⁻¹², 10⁻¹³ M or any range or value therein.

An anti-tissue factor antibody of the present invention can include one or more amino acid substitutions, deletions or additions, either from natural mutations or human manipulation, as specified herein.

Anti-tissue factor antibodies of the present invention can include, but are not limited to, at least one portion, sequence or combination selected from 5 to all of the contiguous amino acids of at least one of SEQ ID NOS: 2 and 4. An anti-tissue factor antibody can further optionally comprise a polypeptide of at least one of 70-100% of the contiguous amino acids of at least one of SEQ ID NOS: 2 and 4 and an Fc portion.

Exemplary heavy chain and light chain variable regions sequences are provided as residues 1-117 of SEQ ID NO: 2 and residues 1-108 of SEQ ID NO: 4. The antibodies of the present invention, or specified variants thereof, can comprise any number of contiguous amino acid residues from an antibody of the present invention, wherein that number is selected from the group of integers consisting of from 10-100% of the number of contiguous residues in an anti-TF antibody. Optionally, this subsequence of contiguous amino acids is at least about 10, 20, 30, 40, 50, 60, 70, 80, 90, 100, 110, 120 or more amino acids in length, or any range or value therein. Further, the number of such subsequences can be any integer selected from the group consisting of from 1 to 20, such as at least 2, 3, 4, or 5.

The carbohydrate structures of all naturally produced antibodies at conserved positions in the heavy chain constant regions varies with isotype. Each isotype possesses a distinct array of N-linked oligosaccharide structures, which variably affect protein assembly, secretion or functional activity (Wright, A., and Morrison, S. L., Trends Biotech. 15:26-32 (1997)). The structure of the attached N-linked oligosaccharides varies considerably, varies with pre- and post-secretory processing, and can be a complex biantennary oligosaccharide structure with or without bisecting GlcNAc and core fucose residues (Wright, A., and Morrison, S. L., supra). Typically, there is heterogeneous processing of the core oligosaccharide structures attached at a particular glycosylation site such that even monoclonal antibodies exist as multiple glycoforms. Likewise, it has been shown that major differences in antibody glycosylation occur between antibody-producing cell lines, and even minor differences are seen for a given cell line grown under different culture conditions.

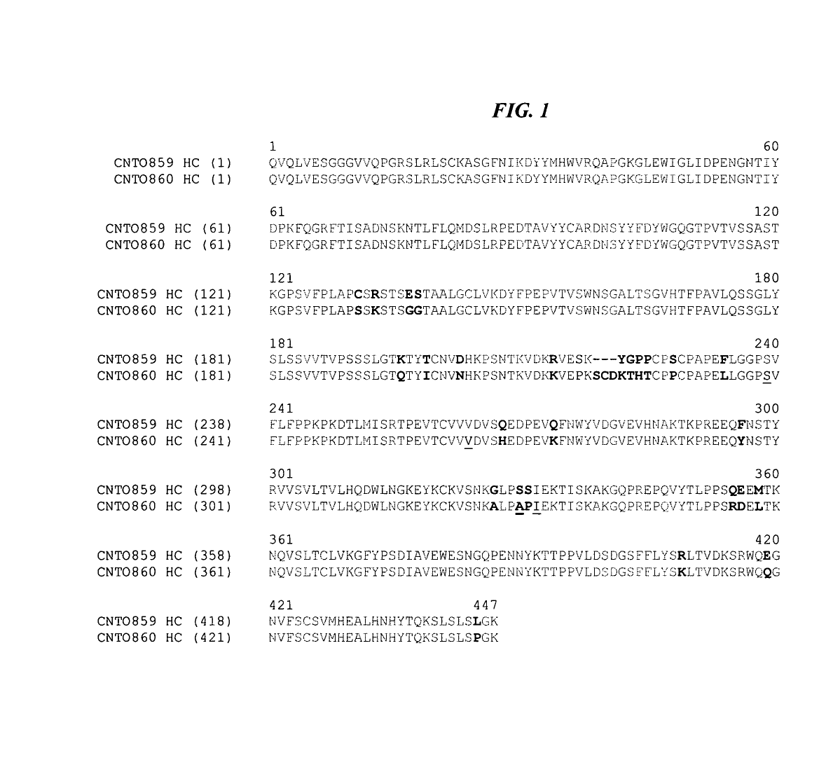

The N-linked oligosaccharides present in the Fc region (formed by the dimerization of the hinge, CH2 and CH3 domains) affect the effector functions. The covalently bound oligosaccharides are complex biantennary type structures and are highly heterogeneous. The CH2 domain of all IgG subtypes contains the unique conserved N-glycosylation site at residue 297 (FIG. 1, SEQ ID NO: 2). In the mature antibody, the two complex bi-antennary oligosaccharides attached to Asn297 are buried between the CH2 domains, forming extensive contacts with the polypeptide backbone. It has been found that their presence is essential for the antibody to mediate effector functions, such as ADCC (Lifely, M. R., et al., Glycobiology 5:813-822 (1995); Jefferis, R., et al., Immunol Rev. 163:59-76 (1998); Wright, A. and Morrison, S. L., 1997 supra).

The presence or absence of glycan in the Fc-containing molecule affects the affinity for one or more of the FcγRI, FcγRIIA, and FcγRIIIA receptors, ADCC activity, macrophage or monocyte activation, and serum half-life (Lifely et al., Jeffreis, and Wright and Morrison, 1997 supra). Recombinant production of antibodies and MIMETIBODY™ constructs by eukaryotic cells will affect the decoration of final composition with a glycan structure typical of the host cell and which glycan structure may be further influenced by the cell culture conditions. These heterogeneous oligosaccharides contain predominantly sialic acid, fucose, galactose and GlcNAc residues as terminal sugars (Raju, T. S., et al. Glycobiology 2000. 10(5): 477-86). It has been shown that some of these terminal sugars, such as exposed galactose, core fucose and bisecting GlcNAc residues, affect ADCC activity, CDC activity, and also affect the antibody binding to various ligands including Clq complement protein (Presta L. 2003. Curr Opin Struct Biol. 13(4): δ 19-25).

In another aspect, the invention relates to antibodies and variants, as described herein, which are modified by the covalent attachment of an organic moiety. Such modification can produce an antibody or antigen-binding fragment with improved pharmacokinetic properties (e.g., increased in vivo serum half-life). The organic moiety can be a linear or branched hydrophilic polymeric group, fatty acid group, or fatty acid ester group. In particular embodiments, the hydrophilic polymeric group can have a molecular weight of about 800 to about 120,000 Daltons and can be a polyalkane glycol (e.g., polyethylene glycol (PEG), polypropylene glycol (PPG)), carbohydrate polymer, amino acid polymer or polyvinyl pyrolidone, and the fatty acid or fatty acid ester group can comprise from about eight to about forty carbon atoms. With respect to the tertiary structure affected by the hinge core interchain disulfide bonds along with the attached glycan, the Fc-containing proteins represent a unique challenge for PEGylation because non-antigen binding properties of antibody reside in the Fc. Thus, a conjugation method that does not significantly impact the glycan structure or the ability of the heavy chain polypeptides to form interchain disulfide bonds is important in the PEGylation of Fc-containing proteins in order to retain biologic activity of the final composition in vivo.

The modified antibodies and antigen-binding fragments of the invention can comprise one or more organic moieties that are covalently bonded, directly or indirectly, to the antibody. Each organic moiety that is bonded to an antibody or antigen-binding fragment of the invention can independently be a hydrophilic polymeric group, a fatty acid group or a fatty acid ester group. As used herein, the term “fatty acid” encompasses mono-carboxylic acids and di-carboxylic acids. A “hydrophilic polymeric group,” as the term is used herein, refers to an organic polymer that is more soluble in water than in octane. Hydrophilic polymers suitable for modifying antibodies of the invention can be linear or branched and include, for example, polyalkane glycols (e.g., PEG, monomethoxy-polyethylene glycol (mPEG), PPG and the like), carbohydrates (e.g., dextran, cellulose, oligosaccharides, polysaccharides and the like), polymers of hydrophilic amino acids (e.g., polylysine, polyarginine, polyaspartate and the like), polyalkane oxides (e.g., polyethylene oxide, polypropylene oxide and the like) and polyvinyl pyrolidone. Preferably, the hydrophilic polymer that modifies the antibody of the invention has a molecular weight of about 800 to about 150,000 Daltons as a separate molecular entity. For example, PEG₅₀₀₀ and PEG_{20,000}, wherein the subscript is the average molecular weight of the polymer in Daltons, can be used. The hydrophilic polymeric group can be substituted with one to about six alkyl, fatty acid or fatty acid ester groups. Hydrophilic polymers that are substituted with a fatty acid or fatty acid ester group can be prepared by employing suitable methods.

Fatty acids and fatty acid esters suitable for modifying antibodies of the invention can be saturated or can contain one or more units of unsaturation. Fatty acids that are suitable for modifying antibodies of the invention include, for example, n-dodecanoate (C₁₂, laurate), n-tetradecanoate (C₁₄, myristate), n-octadecanoate (C₁₈, stearate), n-eicosanoate (C₂₀, arachidate), n-docosanoate (C₂₂, behenate), n-triacontanoate (C₃₀), n-tetracontanoate (C₄₀), cis-Δ9-octadecanoate (C₁₈, oleate), all cis-Δ5,8,11,14-eicosatetraenoate (C₂₀, arachidonate), octanedioic acid, tetradecanedioic acid, octadecanedioic acid, docosanedioic acid, and the like. Suitable fatty acid esters include mono-esters of dicarboxylic acids that comprise a linear or branched lower alkyl group. The lower alkyl group can comprise from one to about twelve, preferably one to about six, carbon atoms.

The modified human antibodies and antigen-binding fragments can be prepared using suitable methods, such as by reaction with one or more modifying agents. A “modifying agent” as the term is used herein, comprises an activating group. An “activating group” is a chemical moiety or functional group that can, under appropriate conditions, react to form a covalent bond between the modifying agent and an antibody or a second organic molecule, such as a linking moeity. For example, amine-reactive activating groups include electrophilic groups such as tosylate, mesylate, halo (chloro, bromo, fluoro, iodo), N-hydroxysuccinimidyl esters (NHS), and the like. Activating groups that can react with thiols include, for example, maleimide, iodoacetyl, acrylolyl, pyridyl disulfides, 5-thiol-2-nitrobenzoic acid thiol (TNB-thiol), and the like. An aldehyde functional group can be coupled to amine- or hydrazide-containing molecules, and an azide group can react with a trivalent phosphorous group to form phosphoramidate or phosphorimide linkages. Suitable methods to introduce activating groups into molecules are known in the art (see for example, Hermanson, G. T., Bioconjugate Techniques, Academic Press: San Diego, Calif. (1996)). A linker moiety, for example, may be a divalent C₁-C₁₂ group wherein one or more carbon atoms can be replaced by a heteroatom, such as oxygen, nitrogen or sulfur. Suitable linker moieties include, for example, a flexible peptide (GGGSₙ), —(CH₂)₃—, —NH—(CH₂)₆—NH—, —(CH₂)₂—NH— and —CH₂—O—CH₂—CH₂—O—CH₂—CH₂—O—CH—NH—.

The modified antibodies of the invention can be produced by reacting an antibody or antigen-binding fragment with a modifying agent. For example, the organic moieties can be bonded to the antibody in a non-site specific manner by employing an amine-reactive modifying agent, for example, an NHS ester of PEG.

It is known in the preparation of conjugates of two substances, of which at least one comprises a protein or a polypeptide, to use bifunctional agents in order to couple the components of the conjugate covalently, amino groups in the conjugated molecules normally being utilized for the conjugating reaction. Bifunctional protein coupling agents include N-succinimidyl-(2-pyridyldithio)propionate (SPDP), succinimidyl-4-(N-maleimidomethyl)cyclohexane-1-carboxylate, iminothiolane (IT), bifunctional derivatives of imidoesters such as dimethyl adipimidate•HCl, active esters such as disuccinimidyl suberate, aldehyes such as glutaraldehyde, bis-azido compounds suc has bis(p-axidobenzoyl)hexanediamine, bis-diazonium derivatives such as bis-(p-diazoniumbenzoyl)-ethylenediamine), diisocyanates (such as tolyene 2,6-diisocyanate), and bis active fluorine compounds such as 1,5-difluoro-2,4-dinitrobenzene). SPDP is among the most frequently used reagent for this purpose and many other N-succinimidyl-(2-pyridyldithio)-, N-succinimidyl-(5-nitro-2-pyridyldithio)- or N-succinimidyl-(4-pyridyldithio)-short chain alkane acids have proved useful.

Antibodies may further be convalently modified, conjugated, to an active thereby forming an immunoconjugate. Immunoconjugates are known and have been described in the art. Examples are doxorubicin conjugated Mab BR96 (Braslawsky, et al. Cancer Immunol Immunother 33:367-374, 1991) and pseudomonas exotoxin fused to anti-growth factor antibodies or fragments (Kreitment, et al., Internat. J. Immunopharm. 14(3):465-72, 1992). It is particularly important to choose a highly potent toxin for antibody targeted therapies in which cells at the target site are desired to be destroyed. If the number of tumor-associated antigens on the cancer cell surface is estimated to be 10⁵ molecules/cell, the cytotoxic agents that can be effectively used in these conjugates must have an IC₅₀ value of 10⁻¹⁰-10⁻¹¹ M against target cancer cells. (Chari, R. V. J. Adv. Drug Delivery Rev. 1998, 31, 89-104). Secondly, the drug must either be released upon binding to the target and penetrate the cell or the entire construct must be transported into the cell and toxin cleaved or otherwise activated there. Antibody conjugates of highly toxic maytansines linked by disulfide bond which degrades slowly extracellularly are on exemplary type of construct with these properties (Chari et al., Cancer Res. 52:127-131, 1992; Liu et al., Proc. Natl. Acad. Sci. USA 93:8618-8623, 1996; U.S. Pat. No. 5,208,020).

### 5. Testing of Antibodies

The affinity or avidity of an antibody for an antigen can be determined experimentally using any suitable method. The measured affinity of a particular antibody-antigen interaction can vary if measured under different conditions (e.g., salt concentration, pH). Thus, measurements of affinity and other antigen-binding parameters (e.g., K_{D}, Kₐ, K_{d}) are preferably made with standardized solutions of antibody and antigen, and a standardized buffer, such as the buffer described herein.

Preferably, the antibody or antigen-binding fragment of the invention binds human tissue factor and, thereby partially or substantially neutralizes at least one biological activity of the protein. An antibody, or specified portion or variant thereof, that partially or preferably substantially neutralizes at least one biological activity of at least one tissue factor protein or fragment can bind the protein or fragment and thereby inhibit activities mediated through the binding of tissue factor to its ligand or through other tissue factor-dependent or mediated mechanisms. As used herein, the term “neutralizing antibody” refers to an antibody that can inhibit a tissue factor-dependent activity by about 20-120%, preferably by at least about 10, 20, 30, 40, 50, 55, 60, 65, 70, 75, 80, 85, 90, 91, 92, 93, 94, 95, 96, 97, 98, 99, 100% or more depending on the assay. The capacity of an anti-tissue factor antibody to inhibit a tissue factor-dependent activity is preferably assessed by at least one suitable tissue factor protein or receptor assay, as described herein and/or as known in the art.

Table 1 provides a listing of the major classes of human and mouse FcγRs, and shows the important classification into activating and inhibiting receptors (signaling through inhibiting receptors at the same time as activating receptors on the same cell may block the signaling cascade that originated from the activating receptors). These amino acid sequences of these receptors are known.

The antibody variants and other Fc-containing proteins of the invention can be compared for functionality by several well-known in vitro assays. In particular, affinity for members of the FcγRs, FcγRII, and FcγRIII family of Fcγ receptors is of interest. These measurements could be made using recombinant soluble forms of the receptors or cell-associated forms of the receptors. In addition, affinity for FcRn, the receptor responsible for the prolonged circulating half-life of IgGs can be measured, using recombinant soluble FcRn. These assays may be conveniently conducted using direct or indirect detection methods using a solid support, e.g., ELISA or by plasmon surface resonance (BIAcore). Cell-based functional assays, such as ADCC assays and CDC assays, provide insights into the likely functional consequences of particular variant structures. In one embodiment, the ADCC assay is configured so as to have NK cells be the primary effector cell, thereby reflecting the functional effects on the FcγRIIIA receptor. Phagocytosis assays may also be used to compare immune effector functions of different variants, as can assays that measure cellular responses, such as superoxide or inflammatory mediator release. In vivo models can also be used, as, for example, measuring T-cell activation in mice, an activity that is dependent on Fc domains engaging specific ligands such as Fcγ receptors or using a disease model, such as an implanted tumor, to gauge the enhancement of tumor cell destruction as measured by either tumor cell regression (reduction in tumor volume) or slowing of tumor growth.

### 6. Anti-Tissue Factor Antibody Compositions

The present invention also provides at least one CNTO860 antibody variant composition comprising at least one CNTO860 antibody variant as described herein provided in a non-naturally occurring composition, mixture or form. The CNTO860 antibody variant compositions or combinations of the present invention can further comprise at least one of any suitable auxiliary, such as, but not limited to, diluent, binder, stabilizer, buffers, salts, lipophilic solvents, preservative, adjuvant or the like. Pharmaceutically acceptable auxiliaries are preferred. Non-limiting examples of, and methods of preparing such sterile solutions are well known in the art, such as, but limited to, Gennaro, Ed., Remington's Pharmaceutical Sciences, 18^{th} Edition, Mack Publishing Co. (Easton, Pa.) 1990. Pharmaceutically acceptable carriers can be routinely selected that are suitable for the mode of administration, solubility and/or stability of the CNTO860 antibody variant composition as well known in the art or as described herein.

Other excipients, e.g., isotonicity agents, buffers, antioxidants, preservative enhancers, can be optionally and preferably added to the diluent. A polypeptide stabilizing agent, such as trehelose, may be used at known concentrations. A physiologically tolerated buffer is preferably added to provide improved pH control. The formulations can cover a wide range of pHs, such as from about pH 4 to about pH 10, and preferred ranges from about pH 5 to about pH 9, and a most preferred range of about 6.0 to about 8.0. Preferred buffers include histidine and phosphate buffers, most preferably sodium phosphate, particularly phosphate buffered saline (PBS).

Other additives, such as a pharmaceutically acceptable solubilizers like Tween 20 (polyoxyethylene (20) sorbitan monolaurate), Tween 40 (polyoxyethylene (20) sorbitan monopalmitate), Tween 80 (polyoxyethylene (20) sorbitan monooleate), Pluronic F68 (polyoxyethylene polyoxypropylene block copolymers), and PEG (polyethylene glycol) or non-ionic surfactants such as polysorbate 20 or 80 or poloxamer 184 or 188, Pluronic® polyls, other block co-polymers, and chelators, such as EDTA and EGTA, can optionally be added to the formulations or compositions.

Pharmaceutical excipients and additives useful in the present composition include but are not limited to proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. One preferred amino acid is histidine.

Carbohydrate excipients suitable for use in the invention include, for example, monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), myoinositol and the like. Preferred carbohydrate excipients for use in the present invention are mannitol, trehalose, and raffinose.

The CNTO 860 antibody Fc-variant compositions can optionally include a buffer or a pH adjusting agent; typically, the buffer is a salt prepared from an organic acid or base. Representative buffers include organic acid salts, such as salts of citric acid, ascorbic acid, gluconic acid, carbonic acid, tartaric acid, succinic acid, acetic acid, or phthalic acid; Tris, tromethamine hydrochloride, or phosphate buffers. Preferred buffers for use in the present compositions are organic acid salts, such as citrate.

Additionally, the CNTO 860 antibody Fc-variant compositions of the invention can include polymeric excipients/additives, such as polyvinylpyrrolidones, ficolls (a polymeric sugar), dextrates (e.g., cyclodextrins, such as 2-hydroxypropyl-α-cyclodextrin), polyethylene glycols, flavoring agents, antimicrobial agents, sweeteners, antioxidants, antistatic agents, surfactants (e.g., polysorbates such as “TWEEN 20” and “TWEEN 80”), lipids (e.g., phospholipids, fatty acids), steroids (e.g., cholesterol), and chelating agents (e.g., EDTA).

These and additional known pharmaceutical excipients and/or additives suitable for use in the CNTO 860 antibody Fc-variant compositions according to the invention are known in the art, e.g., as listed in “Remington: The Science & Practice of Pharmacy”, 19^{th} ed., Williams & Williams, (1995), and in the “Physician's Desk Reference”, 52^{nd} ed., Medical Economics, Montvale, N.J. (1998), the disclosures of which are entirely incorporated herein by reference. Preferred carrier or excipient materials are carbohydrates (e.g., saccharides and alditols) and buffers (e.g., citrate) or polymeric agents.

CNTO 860 antibody Fc-variant compositions of the present invention can optionally further comprise or be combined with the administration of at least one additional agent selected from at least one of: an antirheumatic (e.g., methotrexate, auranofin, aurothioglucose, azathioprine, etanercept, gold sodium thiomalate, hydroxychloroquine sulfate, leflunomide, sulfasalzine), a muscle relaxant, a narcotic, a non-steroid anti-inflammatory drug (NSAID, celecoxib), an analgesic, an anesthetic, a sedative, a local anesthetic, a neuromuscular blocker, an antimicrobial (e.g., aminoglycoside, an antifungal, an antiparasitic, an antiviral, a carbapenem, cephalosporin, a fluororquinolone, a macrolide, a penicillin, a sulfonamide, a tetracycline, another antimicrobial), an antipsoriatic, a corticosteroid, (dexamethasone), an anabolic steroid (testosterone), a agent capable of ameliorating hyperglycemia, a mineral, a nutritional, a thyroid agent, a vitamin, a calcium related hormone, an antidiarrheal, an antitussive, an antiemetic (such as the 5-HT3 inhibitors: dolasetron, granisetron ondansetron, palonosetron), an antiulcer, a laxative, an anticoagulant, an erythropoietin (e.g., epoetin alpha), a filgrastim (e.g., G-CSF, Neupogen), a sargramostim (GM-CSF, Leukine), an immunization, an immunoglobulin (rituximab), an immunosuppressive (e.g., basiliximab, cyclosporine, daclizumab), a growth hormone, a hormone antagonist, a reproductive hormone antagonist (flutamide, nilutamide), a hormone release modulator (leuprolide, goserelin), a hormone replacement drug, an estrogen receptor modulator (tamoxifen), a retinoid (tretinoin), a topoisomerase inhibitor (etoposide, irinotecan), a cytoxin (doxorubicin, dacarbazine), a mydriatic, a cycloplegic, an alkylating agent (cyclophosphamide, chlorambucil), a platinum compound (cisplatin, carboplatin, oxaliplatin, satraplatin), a nitrogen mustard (melphalen, chlorabucil), a nitrosourea (carmustine, estramustine, lomustine) an antimetabolite (methotrexate, cytarabine, fluorouracil, gemcitabine, capcitabine), a mitotic inhibitor (vincristine, taxol, taxoterre, docetaxol), a agent capable of stimulating apoptosis (arsenic trioxide), a signal transduction inhibitor (gefitinib, erlotinib, yolandis, sunitib, imatinib mesylate), a radiopharmaceutical (Iodine131-tositumomab), a radiosensitizer (misonidazole, tirapazamine) an antidepressant, antimanic agent, an antipsychotic, an anxiolytic, a hypnotic, a sympathomimetic, a stimulant, donepezil, tacrine, an asthma medication, a beta agonist, an inhaled steroid, a leukotriene inhibitor, a methylxanthine, a cromolyn, an epinephrine or analog, domase alpha (Pulmozyme), a cytokine (interferon alpha-2, IL2) or a cytokine antagonist (infliximab) where the specified agents and products recited are nonlimiting examples representative of the class or mechanism of action. Suitable agents and dosages are well known in the art. See, e.g., Brunton, et al. (Eds.) Goodman and Gilman's The Pharmacological Basis of Therapeutics, 11^{th} Edition (2006), McGraw-Hill, NY, N.Y. and available online; Wells et al., eds., Pharmacotherapy Handbook, 2^{nd} Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are entirely incorporated herein by reference.

The method may be carried out by combining the administration of the CNTO860 antibody variants of the invention with one or more other agents having anti-tumor effect or a dissimilar mechanism of inhibiting in vivo tumor growth, including, but not limited to chemotherapeutic agents.

Further, CNTO860 antibody variants of the invention can be combined with one or more anti-angiogenic agents such as an anti-vitronectin receptor antibody, e.g., etaracizumab or CNTO95 or as disclosed in U.S. Pat. Nos. 5,985,278 and 6,160,099; U.S. Pat. No. 5,766,591 and WO0078815 and applicants co-pending application published as WO02012501; an anti-VEGF or anti-VEGFR antibody, e.g. bevacizumab (AVASTIN), or nonbiologic agents such as thalidomide. Angiogenisis is known to play a role in various conditions or disease states including tumor metastasis, solid tumor growth (neoplasia), osteoporosis, Paget's disease, humoral hypercalcemia of malignancy, angiogenesis, including tumor angiogenesis, retinopathy, including macular degeneration, arthritis, including rheumatoid arthritis, periodontal disease, psoriasis and smooth muscle cell migration (e.g., restenosis).

The invention also pertains to immunoconjugates comprising the antibody described herein conjugated to a cytotoxic agent such as a chemotherapeutic agent, toxin (e.g., an enzymatically active toxin of bacterial, fungal, plant or animal origin, or fragments thereof), or a radioactive isotope (i.e., a radioconjugate).

Such anti-cancer can also include toxin molecules that are associated, bound, co-formulated or co-administered with at least one antibody of the present invention. The toxin can optionally act to selectively kill the pathologic cell or tissue. The pathologic cell can be a cancer or other cell. Such toxins can be, but are not limited to, purified or recombinant toxin or toxin fragment comprising at least one functional cytotoxic domain of toxin, e.g., selected from at least one of ricin, diphtheria toxin, a venom toxin, or a bacterial toxin. The term toxin also includes both endotoxins and exotoxins produced by any naturally occurring, mutant or recombinant bacteria or viruses which may cause any pathological condition in humans and other mammals, including toxin shock, which can result in death. Such toxins may include, but are not limited to, enterotoxigenic E. coli heat-labile enterotoxin (LT), heat-stable enterotoxin (ST), Shigella cytotoxin, Aeromonas enterotoxins, toxic shock syndrome toxin-1 (TSST-1), Staphylococcal enterotoxin A (SEA), B (SEB), or C (SEC), Streptococcal enterotoxins and the like. Such bacteria include, but are not limited to, strains of a species of enterotoxigenic E. coli (ETEC), enterohemorrhagic E. coli (e.g., strains of serotype 0157:H7), Staphylococcus species (e.g., Staphylococcus aureus, Staphylococcus pyogenes), Shigella species (e.g., Shigella dysenteriae, Shigella flexneri, Shigella boydii, and Shigella sonnei), Salmonella species (e.g., Salmonella typhi, Salmonella cholera-suis, Salmonella enteritidis), Clostridium species (e.g., Clostridium perfringens, Clostridium dificile, Clostridium botulinum), Camphlobacter species (e.g., Camphlobacter jejuni, Camphlobacter fetus), Heliobacter species, (e.g., Heliobacter pylori), Aeromonas species (e.g., Aeromonas sobria, Aeromonas hydrophila, Aeromonas caviae), Pleisomonas shigelloides, Yersina enterocolitica, Vibrios species (e.g., Vibrios cholerae, Vibrios parahemolyticus), Klebsiella species, Pseudomonas aeruginosa, and Streptococci. See, e.g., Stein, ed., INTERNAL MEDICINE, 3rd ed., pp 1-13, Little, Brown and Co., Boston, (1990); Evans et al., eds., Bacterial Infections of Humans: Epidemiology and Control, 2d. Ed., pp 239-254, Plenum Medical Book Co., New York (1991); Mandell et al, Principles and Practice of Infectious Diseases, 3d. Ed., Churchill Livingstone, New York (1990); Berkow et al, eds., The Merck Manual, 16th edition, Merck and Co., Rahway, N.J., 1992; Wood et al, FEMS Microbiology Immunology, 76:121-134 (1991); Marrack et al, Science, 248:705-711 (1990), the contents of which references are incorporated entirely herein by reference.

Conjugates of the antibody and cytotoxic agent are made using a variety of bifunctional protein coupling agents such as N-succinimidyl (2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bisdiazonium derivatives (such as bis-(p-diazoniumbenzoyl)-ethylenediamine), diisocyanates (such as tolyene 2,6diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon labeled 1-isothiocyanatobenzyl methyldiethylene triaminepentaacetic acid (MX-DTPA) is an exemplary chelating agent for conjugation of radionucleotide to the antibody. See WO94/11026.

### 7. Preparations and Articles of Manufacture

As noted above, the invention provides for stable formulations, which is preferably a saline or a chosen salt solution, as well as preserved solutions and formulations containing a preservative as well as multi-use preserved formulations suitable for pharmaceutical or veterinary use, comprising at least one anti-tissue factor antibody in a pharmaceutically acceptable formulation. Antibodies or their binding fragments to be used for in vivo administration must be sterile. This is readily accomplished by filtration through sterile filtration membranes, prior to or following lyophilization and reconstitution. The antibodies, or binding fragments thereof, ordinarily will be stored in lyophilized form or in solution.

As noted above, the invention provides an article of manufacture, comprising packaging material and at least one vial comprising a solution of at least one anti-tissue factor subunit antibody with the prescribed buffers and/or preservatives, optionally in an aqueous diluent, wherein said packaging material comprises a label that indicates that such solution can be held over a period of hours or greater. The invention further comprises an article of manufacture, comprising packaging material, a first vial comprising lyophilized at least one anti-tissue factor antibody, and a second vial comprising an aqueous diluent of prescribed buffer or preservative, wherein said packaging material comprises a label that instructs a patient to reconstitute the at least one anti-tissue factor antibody in the aqueous diluent to form a solution that can be held over a period of twenty-four hours or greater.

The range of tissue factor antibody in the product of the present invention includes amounts yielding upon reconstitution, if in a wet/dry system, concentrations from about 1.0 μg/ml to about 1000 mg/ml, although lower and higher concentrations are operable and are dependent on the intended delivery vehicle, e.g., solution formulations will differ from transdermal patch, pulmonary, transmucosal, or osmotic or micro pump methods.

More specifically, therapeutic formulations of the antibodies, or binding fragments thereof, are prepared for storage by mixing the antibodies or their binding fragments, having the desired degree of purity, with optional physiologically acceptable carriers, excipients, or stabilizers (Remington's Pharmaceutical Sciences, 17th edition, (Ed.) A. Osol, Mack Publishing Company, Easton, Pa., 1985; Gennaro, Ed., Remington's Pharmaceutical Sciences, 18^{th} Edition, Mack Publishing Co. (Easton, Pa.) 1990), in lyophilized form or in the form of aqueous solutions. Acceptable carriers, excipients or stabilizers are nontoxic to recipients at the dosages and concentrations employed, and include buffers such as phosphate, citrate, and other organic acids; antioxidants including ascorbic acid; low molecular weight (less than about 10 amino acid residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, Pluronics or polyethylene glycol (PEG).

The antibodies, or binding fragments thereof, also may be entrapped in microcapsules prepared, for example, by coacervation techniques or by interfacial polymerization (for example, hydroxymethylcellulose or gelatin-microcapsules and poly-[methylmethacylate] microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, supra.

Therapeutic antibody compositions generally are placed into a container having a sterile access port, for example, an intravenous solution bag or vial having a stopper pierceable by a hypodermic injection needle. The route of administration of the antibodies, or binding fragments thereof, in accordance with the present invention, is in accord with known methods, e.g., injection or infusion by intravenous, intraperitoneal, intramuscular, intrarterial, subcutaneous, intralesional routes, by aerosol or intranasal routes, or by sustained release systems as noted below. The antibodies, or binding fragments thereof, are administered continuously by infusion or by bolus injection. Suitable examples of sustained-release preparations include semipermeable matrices of solid hydrophobic polymers containing the protein, which matrices are in the form of shaped articles, e.g., films, or microcapsules. Examples of sustained-release matrices include polyesters, hydrogels (e.g., poly(2-hydroxyethyl-methacrylate) as described by Langer et al., 1981, J. Biomed. Mater. Res., 15:167-277 and Langer, 1982, Chem. Tech., 12:98-105), or poly(vinylalcohol)], polylactides (U.S. Pat. No. 3,773,919; EP 58,481), copolymers of L-glutamic acid and gamma ethyl-L-glutamate (Sidman et al., 1983, Biopolymers, 22:547-556), non-degradable ethylene-vinyl acetate (Langer et al., supra), degradable lactic acid-glycolic acid copolymers such as the LUPRON DEPOT® (injectable microspheres composed of lactic acid-glycolic acid copolymer and leuprolide acetate), and poly-D-(−)-3-hydroxybutyric acid (EP 133,988).

While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in effectiveness. Rational strategies can be devised for antibody stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Sustained-release antibody compositions also include liposomally entrapped antibodies, or their binding fragments. Liposomes containing the antibodies are prepared by known methods, for example, DE 3,218,121; Epstein et al., 1985, Proc. Natl. Acad. Sci. USA, 82:3688-3692; Hwang et al., 1980, Proc. Natl. Acad. Sci. USA, 77:4030-4034; EP 52,322; EP 36,676; EP 88,046; EP 143,949; EP 142,641; Japanese patent application 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30 mol. % cholesterol, the selected proportion being adjusted for the optimal antibody therapy.

An effective amount of antibody to be employed therapeutically will depend, for example, upon the therapeutic and treatment objectives, the route of administration, the age, condition, and body mass of the patient undergoing treatment or therapy, and auxiliary or adjuvant therapies being provided to the patient. Accordingly, it will be necessary and routine for the practitioner to titer the dosage and modify the route of administration, as required, to obtain the optimal therapeutic effect. A typical daily dosage might range from about 1 mg/kg to up to about 100 mg/kg or more, preferably from about 1 to about 10 mg/kg/day depending on the above-mentioned factors. Typically, the clinician will administer antibody until a dosage is reached that achieves the desired effect. The progress of this therapy is easily monitored by conventional assays.

The claimed formulations can be provided to patients as clear solutions or as dual vials comprising a vial of lyophilized tissue factor antibody that is reconstituted with a second vial containing water, a preservative and/or excipients, preferably a phosphate buffer and/or saline and a chosen salt, in an aqueous diluent. Either a single solution vial or dual vial requiring reconstitution can be reused multiple times and can suffice for a single or multiple cycles of patient treatment and thus can provide a more convenient treatment regimen than currently available.

The present claimed articles of manufacture are useful for administration over a period of immediately to twenty-four hours or greater. Accordingly, the presently claimed articles of manufacture offer significant advantages to the patient. Formulations of the invention can optionally be safely stored at temperatures of from about 2 to about 40° C. and retain the biologically activity of the protein for extended periods of time, thus, allowing a package label indicating that the solution can be held and/or used over a period of 6, 12, 18, 24, 36, 48, 72, or 96 hours or greater.

The claimed products can be provided indirectly to patients by providing to pharmacies, clinics, or other such institutions and facilities, the at least one anti-tissue factor antibody as a dried powder, as single vials containing pre-measured amounts of antibody, or as a sterile solution of antibody. The at least one antibody can be prepared as a solution which can be retrieved one or multiple times for transfer into smaller vials and provided by the pharmacy or clinic to their customers and/or patients.

Recognized devices comprising single vial systems include self injector devices such as “pen-injector” devices for delivery of a solution such as or similar those known in the art: BD Pens, BD Autojector®, Biojector®, Needle-Free Injector®, Intraject®, Medi-Ject®, e.g., as made or developed by Becton Dickensen (Franklin Lakes, N.J., www.bectondickenson.com), Disetronic (Burgdorf, Switzerland, www.disetronic.com; Bioject, Portland, Oreg. (www.bioject.com); National Medical Products, Weston Medical (Peterborough, UK, www.weston-medical.com), Medi-Ject Corp (Minneapolis, Minn., www.mediject.com). Recognized devices comprising a dual vial system include those pen-injector systems for reconstituting a lyophilized drug in a cartridge for delivery of the reconstituted solution such as the HumatroPen®.

The products presently claimed include packaging material. The packaging material provides, in addition to the information required by the regulatory agencies, the conditions under which the product can be used. The packaging material of the present invention provides instructions to the patient to reconstitute the at least one tissue factor antibody in the aqueous diluent to form a solution and to use the solution over a period of 2-24 hours or greater for the two vial, wet/dry, product. For the single vial, solution product, the label indicates that such solution can be used over a period of 2-24 hours or greater. The presently claimed products are useful for human pharmaceutical product use.

Tissue factor antibody in either the stable or preserved formulations or solutions described herein, can be administered to a patient in accordance with the present invention via a variety of delivery routes and methods including SC or IM injection; transdermal, pulmonary, transmucosal, implant, osmotic pump, cartridge, micro pump, or other means appreciated by the skilled artisan, as well-known in the art.

### 8. Therapeutic Applications

The CNTO 860 antibody Fc-variants, which are TF antagonists, of the invention are useful in inhibiting and preventing diseases associated with TF activity. A number of pathologies are improved by treatment with TF antagonists in the method of the present invention through inhibition of one of more biological activities associated with TF and complexes comprising TF. Thus, the antibodies of the present invention or specified variants thereof can be used to effect in a cell, tissue, organ or animal (including mammals and humans), to diagnose, monitor, modulate, treat, alleviate, help prevent the incidence of, or reduce the symptoms of, at least one condition mediated, affected or modulated by TF.

The term “therapeutically effective amount” refers to an amount of a drug effective to treat a disease or disorder in a mammal. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; or relieve to some extent one or more of the symptoms associated with the disorder. “Treatment” refers to both therapeutic treatment and prophylactic or preventative measures. Those in need of treatment include those already with the disorder as well as those in which the disorder is to be prevented.

Among TF related pathologies are various forms of solid primary tumors, diseases associated with angiogenesis and those associated with coagulation such as chronic thromboembolic diseases or disorders associated with fibrin formation including vascular disorders such as deep venous thrombosis; diabetes, arterial thrombosis; stroke; tumor metastasis; rejection of a transplanted organ, tissue or cell; thrombolysis, arteriosclerosis and restenosis following angioplasty, acute and chronic indications such as inflammation, septic shock, septicemia, hypotension, adult respiratory distress syndrome (ARDS), disseminated intravascular coagulopathy (DIC) and other diseases.

Tissue factor, the most potent trigger of the coagulation cascade, is increased in diabetic patients with poor glycemic control and circulating tissue factor microparticles are also associated with apoptosis of plaque macrophages, thus forming a link among inflammation, plaque rupture, and blood thrombogenicity in diabetic patients as well as between diabetes and atherosclerosis.

Patients with rheumatoid arthritis (RA) have a two to five times increased risk of developing premature cardiovascular disease that shortens life expectancy by 5-10 years. Similarities exist between the inflammation in the pathogenesis of atherosclerosis and the well-established mechanisms of inflammation in the pathogenesis of RA. Coagulation factors, such as increased levels of TF, van Willebrand factor and plasminogen activator inhibitor-(PAI-)1, are important in both, RA and coronary artery disease. Recent studies have demonstrated impaired endothelial function in patients with RA, already at early stages of the disease. Patients with systemic lupus erythematosus (SLE) similarly display indications that inflammation per se may impair vascular function. Thus, treatment of subjects with RA and SLE may benefit from anti-coagulation therapy as well as a reduction in the activation of TF at the cell surface by the CNTO 860 antibody Fc-variant of the invention.

The association between thrombosis and malignant disease has been known for centuries (Trousseau, et al. Lectures on clinical medicine, R Hardwicke, London (1867). Both benign and malignant tumors, including various cancers, such as, cervical, anal and oral cancers, stomach, colon, bladder, rectal, liver, pancreatic, lung, breast, cervix uteri, corpus uteri, ovary, prostate, testis, renal, brain/cns (e.g., gliomas), head and neck, eye or ocular, throat, skin melanoma, acute lymphocytic leukemia, acute myelogenous leukemia, Ewing's Sarcoma, Kaposi's Sarcoma, basal cell carinoma and squamous cell carcinoma, small cell lung cancer, choriocarcinoma, rhabdomyosarcoma, angiosarcoma, hemangioendothelioma, Wilms Tumor, neuroblastoma, mouth/pharynx, esophageal, larynx, thyroid, kidney and lymphoma, among others may be treated using anti-TF antibodies of the present invention. Clinical manifestations of thromboembolic disease in cancer include deep venous thrombosis, thrombophlebitis, pulmonary embolism, disseminated intravascular coagulation, portal vein thrombosis, and arterial thromboembolism.

Thus, the present invention provides a method for modulating or treating at least one malignant disease, or pathology associated with malignant disease (e.g., thromboembolic complications), in a cell, tissue, organ, animal or patient, including, but not limited to, at least one of: acute promyelocytic leukemia, acute myeloid leukemia (AML), multiple myeloma and Waldenstrom's macroglobulinemia, breast carcinoma, colorectal carcinoma, renal cell carcinoma, pancreatic carcinoma, prostatic carcinoma, nasopharyngeal carcinoma, malignant histiocytosis, paraneoplastic syndrome/hypercalcemia of malignancy, solid tumors, adenocarcinomas, sarcomas, malignant melanoma, hemangioma, metastatic disease, and the like. Such a method can optionally be used in combination with, by administering before, concurrently or after administration of such TF antagonist, radiation therapy which is delivered by external beam, a source placed internally, or administered as a radioisotope containing composition; photodynamic therapy; or the TF antagonist may be administered in conjunction with an additional therapeutic agent or an agent which represents an adjunctive form of care. Therapeutic agents suitable in an anti-neoplastic composition for treating cancer include, but not limited to, chemotherapeutic agents, radioactive isotopes, toxins, cytokines such as interferons, hormones and hormone antagonists, and antagonistic agents targeting cytokines, cytokine receptors or antigens associated with tumor cells.

### Immune Related Disease

The present invention also provides a method for modulating or treating at least one immune related disease, in a cell, tissue, organ, animal, or patient including, but not limited to, at least one of rheumatoid arthritis, juvenile rheumatoid arthritis, systemic onset juvenile rheumatoid arthritis, psoriatic arthritis, ankylosing spondilitis, gastric ulcer, seronegative arthropathies, osteoarthritis, inflammatory bowel disease, ulcerative colitis, systemic lupus erythematosis, antiphospholipid syndrome, iridocyclitis/uveitis/optic neuritis, idiopathic pulmonary fibrosis, systemic vasculitis/wegener's granulomatosis, sarcoidosis, orchitis/vasectomy reversal procedures, allergic/atopic diseases, asthma, allergic rhinitis, eczema, allergic contact dermatitis, allergic conjunctivitis, hypersensitivity pneumonitis, transplants, organ transplant rejection, graft-versus-host disease, systemic inflammatory response syndrome, sepsis syndrome, gram positive sepsis, gram negative sepsis, culture negative sepsis, fungal sepsis, neutropenic fever, urosepsis, meningococcemia, trauma/hemorrhage, burns, ionizing radiation exposure, acute pancreatitis, adult respiratory distress syndrome, rheumatoid arthritis, alcohol-induced hepatitis, chronic inflammatory pathologies, sarcoidosis, Crohn's pathology, sickle cell anemia, diabetes, nephrosis, atopic diseases, hypersensitity reactions, allergic rhinitis, hay fever, perennial rhinitis, conjunctivitis, endometriosis, asthma, urticaria, systemic anaphalaxis, dermatitis, pernicious anemia, hemolytic disesease, thrombocytopenia, graft rejection of any organ or tissue, kidney translplant rejection, heart transplant rejection, liver transplant rejection, pancreas transplant rejection, lung transplant rejection, bone marrow transplant (BMT) rejection, skin allograft rejection, cartilage transplant rejection, bone graft rejection, small bowel transplant rejection, fetal thymus implant rejection, parathyroid transplant rejection, xenograft rejection of any organ or tissue, allograft rejection, anti-receptor hypersensitivity reactions, Graves disease, Raynoud's disease, type B insulin-resistant diabetes, asthma, myasthenia gravis, antibody-meditated cytotoxicity, type III hypersensitivity reactions, systemic lupus erythematosus, POEMS syndrome (polyneuropathy, organomegaly, endocrinopathy, monoclonal gammopathy, and skin changes syndrome), polyneuropathy, organomegaly, endocrinopathy, monoclonal gammopathy, skin changes syndrome, antiphospholipid syndrome, pemphigus, scleroderma, mixed connective tissue disease, idiopathic Addison's disease, diabetes mellitus, chronic active hepatitis, primary billiary cirrhosis, vitiligo, vasculitis, post-MI cardiotomy syndrome, type IV hypersensitivity, contact dermatitis, hypersensitivity pneumonitis, allograft rejection, granulomas due to intracellular organisms, drug sensitivity, metabolic/idiopathic, Wilson's disease, hemachromatosis, alpha-1-antitrypsin deficiency, diabetic retinopathy, hashimoto's thyroiditis, osteoporosis, hypothalamic-pituitary-adrenal axis evaluation, primary biliary cirrhosis, thyroiditis, encephalomyelitis, cachexia, cystic fibrosis, neonatal chronic lung disease, chronic obstructive pulmonary disease (COPD), familial hematophagocytic lymphohistiocytosis, dermatologic conditions, psoriasis, alopecia, nephrotic syndrome, nephritis, glomerular nephritis, acute renal failure, hemodialysis, uremia, toxicity, preeclampsia, OKT3 therapy, anti-CD3 therapy, cytokine therapy, chemotherapy, radiation therapy (e.g., including but not limited toasthenia, anemia, cachexia, and the like), chronic salicylate intoxication, and the like. See, e.g., the Merck Manual, 12th-17th Editions, Merck & Company, Rahway, N.J. (1972, 1977, 1982, 1987, 1992, 1999), Pharmacotherapy Handbook, Wells et al., eds., Second Edition, Appleton and Lange, Stamford, Conn. (1998, 2000), each entirely incorporated by reference.

### Cardiovascular Disease

The present invention also provides a method for modulating or treating at least one cardiovascular disease in a cell, tissue, organ, animal, or patient, including, but not limited to, at least one of cardiac stun syndrome, myocardial infarction, congestive heart failure, stroke, ischemic stroke, hemorrhage, arteriosclerosis, atherosclerosis, restenosis, diabetic ateriosclerotic disease, hypertension, arterial hypertension, renovascular hypertension, syncope, shock, syphilis of the cardiovascular system, heart failure, cor pulmonale, primary pulmonary hypertension, cardiac arrhythmias, atrial ectopic beats, atrial flutter, atrial fibrillation (sustained or paroxysmal), post perfusion syndrome, cardiopulmonary bypass inflammation response, chaotic or multifocal atrial tachycardia, regular narrow QRS tachycardia, specific arrythmias, ventricular fibrillation, H is bundle arrythmias, atrioventricular block, bundle branch block, myocardial ischemic disorders, coronary artery disease, angina pectoris, myocardial infarction, cardiomyopathy, dilated congestive cardiomyopathy, restrictive cardiomyopathy, valvular heart diseases, endocarditis, pericardial disease, cardiac tumors, aordic and peripheral aneuryisms, aortic dissection, inflammation of the aorta, occulsion of the abdominal aorta and its branches, peripheral vascular disorders, occulsive arterial disorders, peripheral atherlosclerotic disease, thromboangitis obliterans, functional peripheral arterial disorders, Raynaud's phenomenon and disease, acrocyanosis, erythromelalgia, venous diseases, venous thrombosis, varicose veins, arteriovenous fistula, lymphederma, lipedema, unstable angina, reperfusion injury, post pump syndrome, ischemia-reperfusion injury, and the like. Such a method can optionally comprise administering an effective amount of a composition or pharmaceutical composition comprising at least one CNTO 860 antibody Fc-variant to a cell, tissue, organ, animal or patient in need of such modulation, treatment or therapy.

### Infectious Disease

The present invention also provides a method for modulating or treating at least one infectious disease in a cell, tissue, organ, animal or patient, including, but not limited to, at least one of: acute or chronic bacterial infection, acute and chronic parasitic or infectious processes, including bacterial, viral and fungal infections, HIV infection/HIV neuropathy, meningitis, hepatitis (A,B or C, or the like), septic arthritis, peritonitis, pneumonia, epiglottitis, e. coli 0157:h7, hemolytic uremic syndrome/thrombolytic thrombocytopenic purpura, malaria, dengue hemorrhagic fever, leishmaniasis, leprosy, toxic shock syndrome, streptococcal myositis, gas gangrene, mycobacterium tuberculosis, mycobacterium avium intracellulare, pneumocystis carinii pneumonia, pelvic inflammatory disease, orchitis/epidydimitis, legionella, lyme disease, influenza a, epstein-barr virus, vital-associated hemaphagocytic syndrome, vital encephalitis/aseptic meningitis, and the like.

Any method of the present invention can comprise administering an effective amount of a composition or pharmaceutical composition comprising at least one of the TF antagonists of the invention are useful in inhibiting and preventing tumor growth. A number of pathologies involving various forms of solid primary tumors are improved by treatment with TF antagonists in the method of the present invention.

### Dosage

Typically, treatment of pathologic conditions is effected by administering an effective amount or dosage of at least one CNTO 860 antibody Fc-variant composition that total, on average, a range from at least about 0.01 to 500 milligrams of at least one tissue factor antibody per kilogram of patient per dose, and preferably from at least about 0.1 to 100 milligrams antibody/kilogram of patient per single or multiple administration, depending upon the specific activity of contained in the composition. Alternatively, the effective serum concentration can comprise 0.1-5000 μg/ml serum concentration per single or multiple administration. Suitable dosages are known to medical practitioners and will, of course, depend upon the particular disease state, specific activity of the composition being administered, and the particular patient undergoing treatment. In some instances, to achieve the desired therapeutic amount, it can be necessary to provide for repeated administration, i.e., repeated individual administrations of a particular monitored or metered dose, where the individual administrations are repeated until the desired daily dose or effect is achieved.

Alternatively, the dosage administered can vary depending upon known factors, such as the pharmacodynamic characteristics of the particular agent, and its mode and route of administration; age, health, and weight of the recipient; nature and extent of symptoms, kind of concurrent treatment, frequency of treatment, and the effect desired. Usually a dosage of active ingredient can be about 0.1 to 100 milligrams per kilogram of body weight. Ordinarily 0.1 to 50, and preferably 0.1 to 10 milligrams per kilogram per administration or in sustained release form is effective to obtain desired results.

Dosage forms (composition) suitable for internal administration generally contain from about 0.1 milligram to about 500 milligrams of active ingredient per unit or container. In these pharmaceutical compositions the active ingredient will ordinarily be present in an amount of about 0.5-99.999% by weight based on the total weight of the composition.

For parenteral administration, the antibody can be formulated as a solution, suspension, emulsion or lyophilized powder in association, or separately provided, with a pharmaceutically acceptable parenteral vehicle. Examples of such vehicles are water, saline, Ringer's solution, dextrose solution, and 1-10% human serum albumin. Liposomes and nonaqueous vehicles such as fixed oils can also be used. The vehicle or lyophilized powder can contain additives that maintain isotonicity (e.g., sodium chloride, mannitol) and chemical stability (e.g., buffers and preservatives). The formulation is sterilized by known or suitable techniques.

As described, the CNTO 860 antibody Fc-variant of the invention may be administered prior to, concurrent with, or subsequent to the administration of a different active which treats, prevents or ameliorates the side-effects of a disease. In one aspect, the administration of the CNTO 860 antibody Fc-variant of the invention may be in conjunction with the administration of a TNF antagonist (e.g., but not limited to a TNF antibody, such as infliximab, golimulmab, or adalimumab, or fragment, such as certolizumab pegol, a soluble TNF receptor or fragment, fusion proteins thereof such as enteracept, or a small molecule TNF antagonist). In another aspect of the practice of the invention, the CNTO 860 antibody Fc-variant may be administered to in conjunction with another monoclonal antibody therapeutic including but not limited to alemtuzumab, gemtuzumab ozogamicin, rituximab, cetuximab, nimotuzumab, matuzumab, bevacizumab, abciximab, daclizumab, basiliximab, trastuzumab, alemtuzumab, omalizumab, efalizumab, palivizumab, denosumab, tocilizumab (MRA, R1569), or specified fragments, conjugates, or variants thereof.

While having described the invention in general terms, the embodiments of the invention will be further disclosed in the following examples.

### EXAMPLE 1

### Preparation of Isotype-Switched Antibody

For oncology indications it is generally preferable to use a human IgG1 isotype subclass antibody, rather than IgG4, to maximize ADCC and CDC mechanisms of tumor cell killing. The IgG1 version of CNTO 859, an antibody whose variable regions were derived from the antibody known as TF8-5G9 (CDR Grafted Antibody TF8HCDR20×TF8LCDR3 as disclosed in EP0833911B1), is designated CNTO 860 (Published Patent Application US20050220793A1), the contents thereof are completely incorporated by reference.

The CNTO860 heavy chain expression plasmid was prepared by polymerase chain reaction amplification of the CNTO859 heavy chain variable region from plasmid pEe6TF8HCCDR20 (EP0833911B1). For reference, the full lenth sequences of the heavy chains of both CNTO859 (IgG4) and CNTO860 (IgG1) are shown aligned in FIG. 1 with the differences in residues shown in bold. The resulting PCR product was digested with Nco I and Hind III, and cloned into the same restriction sites of the plasmid designated p1340. The resulting vector contained the CNTO859 HC variable region downstream of a part of a mouse immunoglobulin promoter. This vector was digested with Xba I and cloned into vector p730. The resulting expression plasmid, p2401, contained an intact mouse immunoglobulin promoter, the CNTO859 HC variable region, the exons for a human G1 constant region, and the gene for E. coli guanine phosphoribosyl transferase. The HC variable region of p2401 was sequenced, and found to contain no PCR or cloning errors.

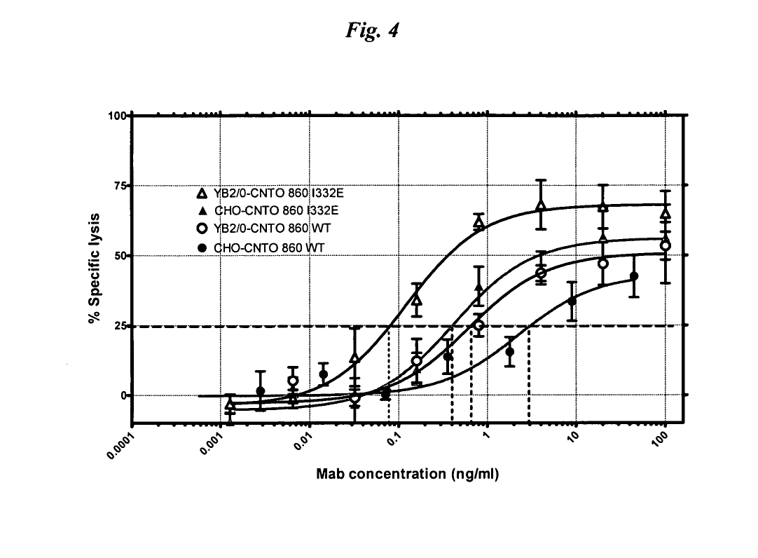

The CNTO860 light chain expression plasmid was prepared by polymerase chain reaction amplification of the CNTO859 light chain variable region from plasmid pEe12TF8LCDR3 (EP0833911B1). The resulting PCR product was digested with Bgl II and Sal I and cloned into the same restriction sites of p2287. The resulting vector contained the CNTO859 LC variable region downstream of a mouse kappa promoter. This vector was digested with Hind III and cloned into vector p95. The resulting expression plasmid, p2402 (FIG. 4), contained a mouse kappa promoter, the CNTO859 LC variable region, a human kappa light chain constant region, and the gene for E. coli guanine phosphoribosyl transferase. The LC variable region of p2402 was sequenced, and found to contain no PCR or cloning errors.

The CNTO860 expression plasmids p2401 and p2402, were transfected into NSO cells for stable expression.

### EXAMPLE 2

### Production of Variants

The effects of specific Fc amino acid substitutions, which substitutions have been disclosed in U.S. Ser. No. 20060483250 to Xencor, Inc., to enhance FcγR binding and ADCC activity of a human IgG1 Abs, of anti-tissue factor Ab, CNTO 860, have been evaluated. In addition to the Fc substitutions specifically described herein, the invention contemplates the use of other Fc substitutions that are described in the patent application cited above and other sources, such as A330L, S298A/E333A/K334A, and S239D/I332E/A330L.

Three single mutant variants (1332E, A330Y, A330J) and three double mutant variants (A330I/I332E, V264I/I332E, S239D/I332E) of CNTO 860 heavy chain (SEQ ID NO: 2) were prepared by mutating DNA encoding CNTO 860 (SEQ ID NO: 1). The amino acid sequence of the hinge and Fc domain of CNTO 860 (unsubstituted sequence is referred to as wild-type or WT) beginning from residue 226 are found in FIG. 1. The variant heavy chain genes were expressed with the normal CNTO 860 light chain (SEQ ID NO: 4) in mammalian cells, either CHO cells via transient transfection, or rat YB2/0 cells by stable transfection. While transient transfection allows more expedient production of product an efficient transient transfection protocol had not been established for the YB2/0 cell line.

Expressing of each CNTO 860 Fc-variant in both CHO and YB2/0 cells causes the resulting antibody to be decorated by glycans (N-linked glycosylation) unique to the host cell. CHO cells typically produce Abs that are 95% fucosylated whereas YB2/0 cells typically produce Abs that are 40-60% fucosylated. Reduced levels of core fucose in the Fc glycan has been shown to enhances ADCC potency. Thus, the pairs of antibodies from different host cells can be evaluated to gauge whether the effects on Fc-mediated bioactivities produced by amino acid changes as well as different glycan structures are subadditive, additive, or synergistic.

### Preparation of Expression Plasmids

Prior to performing DNA mutagenesis, a shuttle vector with more convenient restriction sites was prepared by transferring a 2.4 kb SpeI-HindIII fragment containing all of the human IgG1 constant region coding sequence in cDNA format from a previously-prepared Centocor plasmid, p1483, into the 3.4 kb SpeI-HindIII vector backbone of pBC (Strategene). The resulting plasmid was referred to as p4114. Expression plasmids encoding the heavy chain of each of the six CNTO 860 variants were then constructed in a two-step process. First, desired mutations were introduced into p4114 using the QuikChange II Site-Directed Mutagenesis Kit (Stratagene), the primers listed in Table 3, and plasmid p4114 as template.

TABLE 3
Oligonucleotide primers used for mutagenesis

| | | | |
|---|---|---|---|
| | | | SEQ ID |
| Variant | Primer Name | Primer Sequence | NO |

| | | | |
|---|---|---|---|
| I332E | hG1-ItoE-QC1 | CAAAGCCCTCCCAGCTCCGGAG | 5 |
| | | GAGAAAACCATCTCCAAAGCC | |
| | hG1-ItoE-QC2 | GGCTTTGGAGATGGTTTTCTCC | 6 |
| | | TCCGGAGCTGGGAGGGCTTTG | |
| A3301 | A330I-QC1 | CAACAAAGCCCTCCCAATCCCG | 7 |
| | | ATCGAGAAAACCATCTC | |
| | A330I-QC2 | GAGATGGTTTTCTCGATCGGGA | 8 |
| | | TTGGGAGGGCTTTGTTG | |
| A330Y | A330Y-QC1 | CAACAAAGCCCTCCCATACCCG | 9 |
| | | ATCGAGAAAACCATCTC | |
| | A330Y-QC2 | GAGCTGGTTTTCTCGAGCGGGT | 10 |
| | | ATGGGAGGGCTTTGTTG | |
| A330I/ | I332E/A330I- | CCAACAAAGCCCTCCCAATCCC | 11 |
| I332E | QC1 | GGAGGAGAAAACCATC | |
| | I332E/A330I- | GATGGTTTTCTCCTCCGGGATT | 12 |
| | QC2 | GGGAGGGCTTTGTTGG | |
| V264I/ | I332E/V264I- | GAGGTCACATGCGTGGTCATCG | 13 |
| I332E | QC1 | ATGTGAGCCACGAAGACC | |
| | I332E/V264I- | GGTCTTCGTGGCTCACATCGAT | 14 |
| | QC2 | GACCACGCATGTGACCTC | |
| S239D/ | I332E/S239D- | CTCCTGGGGGGTCCGGACGTCT | 15 |
| I332E | QC1 | TCCTCTTCCCC | |
| | I332E/S239D- | GGGGAAGAGGAAGACGTCCGGA | 16 |
| | QC2 | CCCCCCAGGAG | |

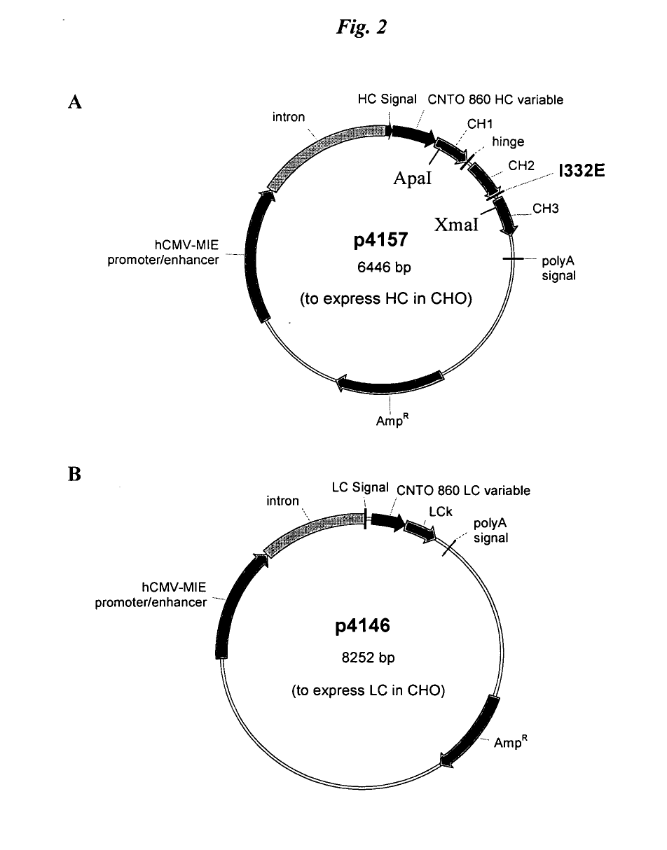

Successful mutagenesis and absence of inadvertent mutations were confirmed by DNA sequencing of the entire constant region coding sequences for each variant. Secondly, a restriction fragment spanning the altered sequence in the mutagenized p4114 shuttle vector was transferred into a final expression vector. In the case of plasmids for CHO cell expression, this entailed transferring a 0.7 kb ApaI-XmaI fragment from the mutagenized p4114 plasmid in place of the corresponding fragment in p4145, an expression plasmid encoding the wild-type version of CNTO 860 heavy chain behind a CMV promoter. One of the resulting plasmids, p4157 encoding the I332E variant, is depicted schematically in FIG. 2, along with the previously-prepared plasmid encoding the CNTO 860 light chain, p4146. The heavy chain plasmids encoding the other variants for CHO expression are listed in Table 4.

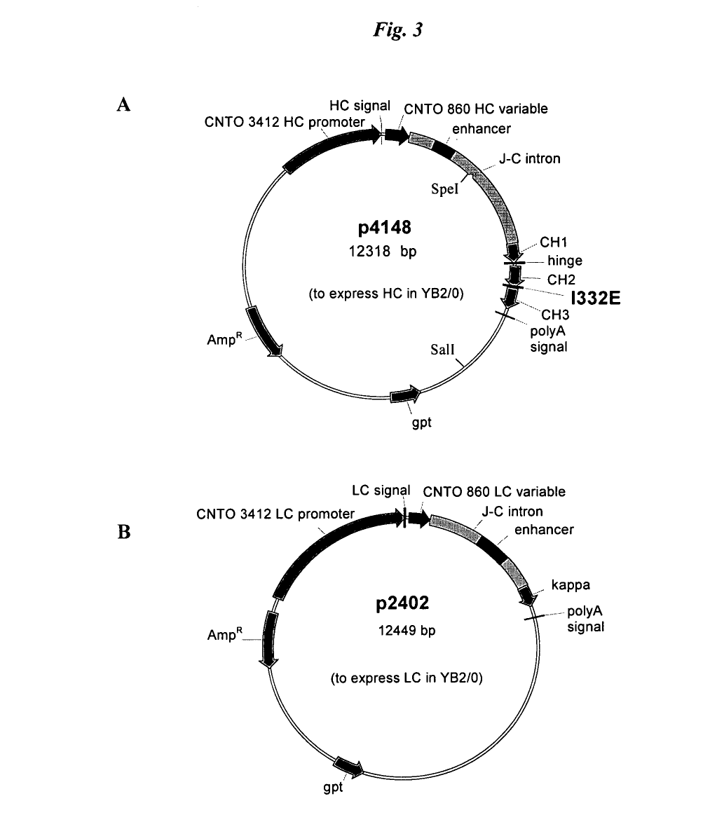

For the YB2/0 cell expression, the same plasmids used for CHO cells were not used due to reports of very low expression of CMV-driven Ab genes in YB2/0 cells. Instead, a 2.3 kb SpeI-SalI fragment from the mutagenized p4114 plasmid was cloned in place of the corresponding fragment in p2401, the previously-prepared expression plasmid encoding the CNTO 860 heavy chain behind the heavy chain gene promoter of antibody CNTO 3412, a high-expressing, mouse/human chimeric anti-human CD4 antibody (Looney J E, et al., 1992, Hum Antibod Hybrid, 3(4): 191). One of the resulting plasmids, p4148 encoding the I332E variant, is depicted schematically in FIG. 3, along with the previously-prepared plasmid encoding the CNTO 860 light chain, p2402, whose expression is driven by a natural immunoglobulin light chain gene promoter. The heavy chain plasmids encoding the other variants for YB2/0 expression are listed in Table 4.

TABLE 4
Expression Plasmid Designations

| Variant | HC Plasmid | LC Plasmid |
|---|---|---|
| CHO-CNTO 860 WT | p4145 | p4146 |
| CHO-CNTO 860 I332E | p4157 | p4146 |
| CHO-CNTO 860 A330I | p4184 | p4146 |
| CHO-CNTO 860 A330Y | p4183 | p4146 |
| CHO-CNTO 860 I332E/A330I | p4185 | p4146 |
| CHO-CNTO 860 I332E/V264I | p4187 | p4146 |
| CHO-CNTO 860 I332E/S239D | p4186 | p4146 |
| YB2/0-CNTO 860 WT | p2401 | p2402 |
| YB2/0-CNTO 860 I332E | p4148 | p2402 |
| YB2/0-CNTO 860 A330I | p4177 | p2402 |
| YB2/0-CNTO 860 A330Y | p4176 | p2402 |
| YB2/0-CNTO 860 A330I/I332E | p4178 | p2402 |
| YB2/0-CNTO 860 V264I/I332E | p4180 | p2402 |
| YB2/0-CNTO 860 S239D/I332E | p4179 | p2402 |

### Expression and Purification of CNTO 860 Variants

Antibodies were expressed from transiently transfected CHO cells and from isolated clones of stably transfected YB2/0 cells. Wild-type transfections were done by co-transfecting the wild type heavy and light chain expression plasmids shown in Table 4 for each appropriate cell host. Variant transfections were done using each variant heavy chain plasmid co-transfected with the host-appropriate wild type light chain plasmid.

Transient transfections of CHO cells were performed using LipofectAMINE reagent (Invitrogen) by standard protocol. Stable YB2/0 transfectants were made by electroporation using a BioRad model at 975 uFD, 0.2 kV. Cells were plated out by limiting dilution and single colonies screened by anti-human IgG (Fc-specific) ELISA. Cell supernatants were obtained from large-scale spent cultures and secreted Abs were purified by protein A using our standard protocol.

### EXAMPLE 3

### Evaluation of Variant Activity

The purified CNTO 860 variants were evaluated for their relative activity at inducing killing (ADCC) of antigen-expressing target cells by peripheral blood mononuclear cells (PBMC). Target cells, HCT116 human colorectal carcinoma cells, were obtained from ATCC and cultured in DMEM-10% heat-inactivated FBS+2 mM L-glutamine, 1 mM sodium pyruvate and 0.1 mM nonessential amino acids. Cells were passaged twice a week and maintained in log phase growth. Culture media and supplements were purchased from Gibco (InVitrogen). On the day of the experiment, cells were removed by trypsinization and washed twice. Cells were adjusted to 1×10⁶ cells/ml with culture medium and 15 ul of BATDA fluorescent labeling reagent (in Delfia EuTDA Cytotoxicity Kit, Perkin-Elmer Life Sciences) was added to 5 ml of cells (Blomberg K, et al., 1996, J. Immunol. Methods 193:199-206). Cells were incubated for 30 min at 37° C. with occasional shaking; then washed twice with media. Immediately prior to mixing with effector cells, targets cells were centrifuged and resuspended at 2×10⁵ cells/ml in culture media. PBMC, the effector cells, were isolated from heparinized blood of healthy donors. Blood samples were diluted with phosphate buffered saline (PBS) and PBMC were isolated by density gradient centrifugation on Ficoll-Hypaque (Amersham). After centrifugation, PBMC were collected, washed twice, and kept overnight in culture media at 37° C. with 5% CO₂. On the following day, PBMC were collected, washed and resuspended in media at 1×10⁷ cells/ml. Antibody dilutions in 100 μl culture media were added to a round bottom 96-well plate. Fifty μl of effector cells and 50 μl of Europium-labeled target cells were added to the Ab dilutions at an effector to target cell ratio of 50:1. The plate was centrifuged briefly to bring effectors and targets in contact with each other, and then incubated for 2 h at 37° C. in a 5% CO₂ atmosphere. After incubation, 20 μl of supernatants were transferred to wells of a flat bottom 96 well plate and 200 μl aliquot of Europium enhancement solution (in Delfia Cytotoxicity kit) was added to each well. After shaking the plate for 10 min, fluorescence was measured in a time-resolved fluorometer (EnVision instrument, Perkin-Elmer). The percentage of specific cytotoxicity was calculated as (experimental release-spontaneous release)/(maximum release−spontaneous release)×100. Spontaneous release was determined by incubating the targets with media instead of effector cells, and maximum release (100% lysis) was determined by incubating the targets with 10 ul of lysis solution containing digitonin (in Delfia EuTDA Cytotoxicity kit). Samples were tested in triplicate and results shown are representative of 2 or 3 independent experiments.

FIG. 4 shows a representative set of curves for the ADCC assay data as performed. For antibody produced in CHO cells, the single mutant CNTO 860 I332E expressed in CHO cells was approximately 7-fold more potent in the ADCC assay, that is CHO-CNTO 860 WT present at 3 ng/ml produced 25% specific lysis whereas CHO-CNTO 860 I332E produced 25% lysis at only about 0.4 ng/ml (FIG. 4). Similarly, the single mutant CNTO 860 I332E expressed in YB2/0 cells was about 8-fold more potent as 0.08 ng/ml produced 25% lysis compared to 0.65 ng/ml for the WT.

In a separate experiment, where all of the variants were compared under the same conditions, the concentration of antibody required to produce 25% lysis was used as a comparator (Table 5) as not all of the tested variants or WT antibody produced 100% lysis.

TABLE 5

| | CHO Expressed | YB2/0 Expressed |
|---|---|---|
| Variant | EC25 (ng/ml) | EC25 (ng/ml) |
| WT | >8.0 | 0.9 |
| I332E | 0.75 | 0.3 |
| A330I | Not Achieved | 0.7 |
| A330Y | 4 | 0.4 |
| A330I/I332E | 1.2 | 0.15 |
| V264I/I332E | 1.0 | 0.12 |
| S239D/I332E | 0.7 | 0.2 |
| WT-deglycosylated | Not Achieved | |

The data shown in Table 5 confirm that the antibody variants expressed in YB2/0 cells are generally about 10-fold more potent in the in vitro ADCC assay than the same variant expressed in the CHO cells. Overall the I332E mutation had the largest magnitude of effect, increasing potency relative to WT expressed in the same host line by 3 to 10-fold in each variant where it was present either singly or with the other mutionations. Among the other CHO cell-expressed mutants, the A330Y single mutant appeared to result in little or no enhancement of ADCC activity and the A330I may have reduced ADCC activity. The three double mutants expressed in CHO cells all showed greater activity than CNTO 860 WT, although the double mutants did not show greater potency than the I332E single mutant.

The I332E variant expressed by YB2/0 cell-expressed also appeared to be the most potent of the three single mutants expressed by this host cell, although to a lesser extent than was observed for the CHO-derived samples. The I332E, A330Y, and A330I variants each showed several-fold greater potency, respectively, than the WT control from YB2/0 cells. The double mutants expressed in YB2/0 cells all showed 5 to 8-fold greater activity than YB2/0-CNTO 860 WT.

These data also serve to show effect of Fc glycan structures independently and in combination with the Fc-region substitution on each variant produced using CHO and YB2/0 host cell lines. Enzymatically deglycosylated CNTO 860 (Gno) produced only baseline values of specific lysis at all concentrations as has been reported previously for deglycosylated antibodies. Importantly, the combination of the I332E mutation and the low-fucose glycan structure (YB2/0 produced material) appeared to have additive or even synergistic effects, since the concentrations needed to achieve 25% lysis was dramatically reduced (0.08 ng/ml) for YB2/0-CNTO 860 I332E, an almost 100-fold reduction in potency from WT CNTO860 from CHO host cells. Besides lowering the amount of Ab required to achieve a particular degree of lysis, the I332E mutation also resulted in a higher maximum lysis, e.g. 70% lysis for YB2/0-CNTO 860 I332E vs 50% lysis for YB2/0-CNTO 860 WT (FIG. 4).

### EXAMPLE 4

### Human Receptor Binding by Variants

The heavy chain CNTO860 antibody Fc-variants produced in Example 2 were used to assess changes in binding to the Fc-domain binding receptors collectively known as Fc receptor gamma types (FcγR) including FcγRI, FcγRII FcγRIII, and FcγRIV. As described above, the receptors can be classified as activating or inhibitory of cell mediated antibody functions.

FcγRI (CD64) ELISA —50 μl of a 1 μg/ml solution of recombinant human His-tagged FcγRI (extracellular domain, R&D Systems #1257-FC-050) in ELISA buffer (PBS, pH 7.4, 4 mg/ml BSA, 0.01% Tween-20) was added to each well of HisGrab 96 well plates (Pierce #15142) (Powers G., Notebook 9006, pp. 166-168, 180-181). The plate was incubated with shaking for 3 hours. The plates were washed 3× with 300 μl of wash buffer (PBS, 0.01% Tween-20) on a plate washer. Serial dilutions of the CNTO860 antibody Fc-variant samples were made in ELISA buffer and 50 μl per well of each titration were added in duplicate to the FcγRI-coated plates. The plates were incubated with shaking for 1 hour. The plates were washed 3× with 300 μl of wash buffer on a plate washer. 50 μl of a 1:10,000 dilution of HRP-labeled goat F(ab′)₂ anti-human IgG F(ab′)₂ (Jackson Immunoresearch # 109-036-097) in ELISA buffer was added to each well. The plates were incubated with shaking for 30 minutes. The plates were washed 3× with 300 μl of wash buffer on the plate washer. 50 μl per well TMB substrate (RDI #RDI-TMBSU-1L) was added to each well and developed for 5 minutes. The reaction was stopped by adding 100 μl 0.2 N sulfuric acid. The plates were read at OD450 on an Envision plate reader.

FcγRIIa (CD32a) AlphaScreen—Nickel acceptor beads (Perkin Elmer #6760619C) were diluted 1:50 in assay buffer (PBS, pH 7.4, 4 mg/ml BSA, 0.01% Tween-20), and 27.5 μl were added to each well of a Nunc V-bottom polypropylene plate (VWR # 62409-108) (Powers G., Notebook 9006, pp. 169-175, 182-185). A 2.0 μg/ml solution of recombinant human His-tagged FcγRIIa (R&D Systems #1330-CD-050) was prepared in assay buffer and 27.5 μl added to each well of a separate Nunc V bottom polypropylene plate. Titrations of test Abs were prepared in the Nunc V bottom polypropylene plate starting at 30 μg/ml. A 2 μg/ml solution of biotinylated goat F(ab′)₂ anti-human IgG F(ab′)₂ (Jackson Immunoresearch #109-066-097) was prepared in assay buffer and 27.5 μl added to each well of another Nunc V bottom polypropylene plate. A 1:50 dilution of streptavidin donor beads (PerkinElmer # 6760002) was prepared in assay buffer and 27.5 μl added to each well of another Nunc V bottom polypropylene plate. A volume of 5 μl of each reagent was added to the corresponding quadrants of a low-volume, non-binding white 384-well plate (Corning # 3673) in the following order: acceptor beads, FcγRIIa, test Ab, biotinylated anti-human IgG F(ab′)₂, and streptavidin donor beads. The plates were incubated for 30 minutes with shaking and the O.D.s determined using an Envision plate reader.

FcγRIIb (CD32b) AlphaScreen—The assay was performed as described above for FcγRIIa except that FcγRIIa was replaced with FcγRIIb (R&D Systems #1875-CD-050).

Binding of the different variants to FcγRIIb showed greater variability than with the other two human receptors (Table 6). The highest affinity binding was by the S239D/I332E double mutant and was about 6-fold enhance over WT. The weakest binding was by the A330I single mutant, a 500-fold reduction in binding affinity. The A330I/I332E double mutant bound FcγRIIa receptor 30-100-fold more weakly than WT, though it bound to FcγRI with the same affinity as WT. Such a receptor binding profile may be of particular interest, since reduced binding to FcγRIIb but high binding to other FcγRs may offer greater therapeutic efficacy, such as when the immune effector cells express both receptor types (e.g. macrophages).

FcγRIIIa (CD16a) ELISA—A human FcγRIIIa binding assay was done in ELISA format similar to the assay for FcγRI above. 50 μl of a 2 μg/ml solution of recombinant human His-tagged FcγRIIIa (extracellular domain made at Centocor) in ELISA buffer (PBS, 4 mg/ml BSA, 0.01% Tween-20) was added to each well of HisGrab 96 well plates (Pierce #15142) (Powers G., Notebook 10378, 19-23). The plate was incubated for 3 hours at room temp. The plates were blocked with 200 μl of Starting Block (Pierce #15142) for 30 min, and then washed 3× with 300 μl of wash buffer (PBS, 0.01% Tween-20) on a plate washer. Serial dilutions of the CNTO 860 test samples were made in Starting Block buffer and 50 μl per well of each titration were added in duplicate to the FcγRIIIa-coated plates. The plates were incubated for 1 hour. The plates were washed 3× with 300 μl of wash buffer on a plate washer. 50 μl of a 1:10,000 dilution of HRP-labeled goat F(ab′)2 anti-human IgG F(ab′)2 (Jackson Immunoresearch # 109-036-097) in Starting Block buffer was added to each well. The plates were incubated for 30 minutes. The plates were washed 3× with 300 μl of wash buffer on the plate washer. 50 μl per well TMB substrate (RDI #RDI-TMBSU-1L) was added to each well and developed for 3-5 minutes. The reaction was stopped by adding 100 μl 10.2 N sulfuric acid. The plates were read at OD450 on an Envision plate reader. Antibody concentration vs OD450 values were graphed for each CNTO 860 variant and the antibody concentration needed to achieve an OD450 reading of 0.14 established by interpolation within each data curve.

In the case of human FcγRI the results showed that the single and double mutations in the heavy chain produced relatively subtle effects on antibody binding affinity, regardless of whether the antibody was expressed in CHO cells or YB2/0 cells. Binding by the YB2/0-derived variants was generally similar to binding by the CHO-derived variants, which is consistent with previous observations that FcγRI is not sensitive to Ab fucose levels. The I332E variant showed enhanced binding by a factor of 2, while the A330I variant appeared to show 2-3-fold reduced binding, and several variants showed the same binding as CNTO 860 WT. A summary of data from all variants analyzed for human FcγRI binding, shown as the amount of Ab required to yield an OD value of 1.0 in the assay, is shown in Table X. Values shown are the concentrations (ng/ml) of the CNTO 860 variant required to result in an O.D. value of 1.0 in the ELISA assay.

TABLE 6
Binding to recombinant, soluble, human FcγRI

| | CHO cell-derived | YB2/0 cell-derived |
|---|---|---|
| WT | 480 | 420 |
| I332E | 300 | 300 |
| A330I | 750 | 570 |
| A330Y | 420 | 420 |
| A330I/I332E | 420 | 310 |
| V264I/I332E | N.D. | 480 |
| S239D/I332E | 480 | 300 |

N.D. = not done

The results from the human FcγRIIa binding analyses (Table 7) showed that, with exception of the I332E single mutant and S239D/I332E double mutant showing similar binding as WT, the variants bound weaker to soluble, recombinant human FcRIIa than did CNTO 860 WT. However, binding by different variants relative to each other followed a similar pattern to what has been reported by publications of Xencor, raising at least the possibility that the binding observed by CNTO 860 WT in these experiments was abnormally high. Interestingly, the A330I/I332E double mutant needed to be present at about 20-fold higher concentrations than WT to give an OD signal of 200,000, even though there was no difference in binding to FcγRI by those two samples. Because FcγRIIa receptor is expressed on platelets in addition to monocytes and macrophages, Ab binding to it should be considered during Ab optimization efforts. Values shown are the concentrations (ng/ml) of the CNTO 860 variant required to result in an O.D. value of 1.0 in the ELISA assay.

TABLE 7x
Binding to recombinant, soluble, human FcγRIIa

| | CHO cell-derived | YB2/0 cell-derived |
|---|---|---|
| WT | 97 | 63 |
| I332E | 76 | 60 |
| A330I | 2250 | 450 |
| A330Y | 142 | 78 |
| A330I/I332E | 900 | 1500 |
| V264I/I332E | N.D. | 90 |
| S239D/I332E | 75 | 60 |

Binding of the different variants to FcγRIIb showed greater variability than with the other two human receptors (Table 8). The tightest binding was enhanced about 6-fold by the S239D/I332E double mutant and the weakest binding was a 500-fold reduced binding by the A330I single mutant. Similar to what was noted with the FcγRIIa receptor, the A330I/I332E double mutant bound 30-100-fold weaker than WT, even though it bound to FcγRI with the same affinity as WT. Such a receptor binding profile may be of particular interest, since reduced binding to FcγRIIb but high binding to other FcγRs may offer greater therapeutic efficacy, such as when the immune effector cells express both receptor types (e.g. macrophages). In Table X, the values shown are the concentrations (ng/ml) of the CNTO 860 variant required to result in an O.D. value of 200,000 in the AlphaScreen assay.

TABLE 8
Binding to recombinant, soluble, human FcγRIIb

| | CHO cell-derived | YB2/0 cell-derived |
|---|---|---|
| WT | 165 | 120 |
| I332E | 105 | 75 |
| A330I | 75000 | 37000 |
| A330Y | 280 | 165 |
| A330I/I332E | 4500 | 10500 |
| V264I/I332E | N.D. | 60 |
| S239D/I332E | 27 | 20 |

The results showed that, with the exception of the A330I single mutant, each variant bound more strongly to FcγRIIIa than CNTO 860 WT. Although complete curves for some samples were not obtained due to a limited supply of material, the I332E variant from CHO cells appeared to bind 6-fold better than WT from CHO cells (Table 9), and I332E from YB2/0 cells bound about 10-fold better than WT from YB2/0 cells (Table 9). The A330I/I332E variant from CHO cells bound about 8-fold better than WT from CHO cells, and the A330I/I332E from YB2/0 cells bound 6-fold better than WT from YB2/0 cells. In general, Ab expressed in YB2/0 cells appears to bind approximately 10-fold better than their CHO-derived counterparts. Because FcγRIIIa is the sole FcγR on NK cells, the primary effector cell in the PBMC population used for the ADCC assays, these binding results should also represent correlate with the ADCC data which was confirmed. In addition, the FcγRIIIa binding data supports the ADCC data in showing an additive/synergistic effect when combining amino acid substitutions with the low-fucose glycan structure from YB2/0 cells. For example, whereas the I332E from CHO cells bound 6-fold better than WT from CHO cells, and the WT from YB2/0 bound about 3-fold better than the WT from CHO, the I332E from YB2/0 cells bound about 30-fold better than WT from CHO. A summary of all samples analyzed for human FcγRIIIa binding is shown in Table 9. Values shown are the concentrations (ng/ml) of the CNTO 860 variant required to result in an O.D. value of 0.14 in the ELISA assay.

TABLE 9x
Binding to recombinant, soluble, human FcγRIIIa

| | CHO cell-derived | YB2/0 cell-derived |
|---|---|---|
| WT | 4500 | 1500 |
| I332E | 750 | 165 |
| A330I | 19000 | 1200 |
| A330Y | 3300 | 340 |
| A330I/I332E | 520 | 240 |
| V264I/I332E | 900 | 165 |
| S239D/I332E | 600 | 120 |

### EXAMPLE 5

### Murine Receptor Binding by Variants

Recombinant murine FcγR was used to assess variations in binding affinity of the CNTO860 antibody Fc-variants produced in Example 2.

Commercially-available recombinant polyhistidine tagged mouse FcγRI, FcγRII, FcγRIII, and FcγRIV (R&D Systems) were diluted to 1 μg/ml in PBS and 100 μl/well was captured onto copper-coated plates (Pierce) overnight at 4° C. Plates were washed three times with wash buffer (0.15M NaCl, 0.02% Tween 20). Non-specific binding was blocked using 200 μl/well SuperBlock (Pierce) for 15 minutes at room temperature. Plates were washed three times as above. CNTO 860 wild type and variant antibodies which had been serially diluted in PBS were allowed to bind at a volume of 100 μl/well at room temperature for 1 hour. Plates were washed three times as above to remove unbound mAb. CNTO 860 variants that bound to plated receptors were detected using 100 μl/well HRP-labeled goat F(ab′)₂ anti-human IgG F(ab′)₂ (Jackson ImmunoResearch) diluted 1:5,000 in PBS incubated at room temperature for 1 hour. Plates were washed five times using wash buffer as above, and developed using 100 μl/well TMB Stable Stop substrate (Fitzgerald Industries) stopped with 0.5M HCl. Absorbance was detected at 450 nm.

The CNTO 860 variants showed variation in murine FcγR binding ranging from 5 to 10-fold higher affinity binding than CNTO 860 WT to 5 to 10-fold weaker binding. When plotted as OD₄₅₀ (bound material) vs concentration, the data for CNTO 860 WT, CNTO 860 I332E, and CNTO 860 A330I/I332E binding to each of the four mouse Fc receptors resulted in a typical sigmoidal binding curve (data not shown).

To summarize and compare the relative binding affinity of each variant, the concentration required to obtain an OD reading of 1.0 (“EC_{OD1}”) was interpolated from the binding curve (presented in Table 10). All binding assays were in an ELISA format, with recombinant soluble receptor captured on coated EIA plates. Values shown are the concentrations (ng/ml) of the CNTO 860 variant required to result in an O.D. value of 1.0. An OD of 1.0 was chosen as these concentrations corresponded to the linear part of the response curve for all samples.

In general, the relative binding by the different variants tended to follow the pattern observed with the human receptors, e.g., the I332E variant binding both FcγRI and FcγRII 2-4-fold greater (human FcγRI and FcγRIIb, respectively), A330Y binding to FcRII about as well as WT, but A3301 binding weaker. One notable exception was the A330I/I332E variant, which bound to the inhibiting mouse receptor FcγRII as strong or stronger than WT, whereas it bound substantially weaker than WT to the inhibiting human receptor, FcγRIIb. Of interest is the observation that the enhanced binding to inhibiting mouse receptor FcRII by I332E relative to WT is similar in magnitude (3-6-fold) to the enhancement observed for mouse FcγRIII and FcγRIV, raising the possibility that the beneficial effect of enhanced binding to the latter activating receptors may be offset by the enhanced binding by the inhibiting receptor when the immune cells express both receptor types.

TABLE 10
Binding to recombinant, soluble, mouse FcγRs

| | | | |
|---|---|---|---|
| FcγRI | FcγRII | FcγRIII | FcγRIV |

CHO-derived CNTO 860 variants

| | | | | |
|---|---|---|---|---|
| WT | N.D. | 4000 | 4000 | 60 |
| I332E | 4000 | 1500 | 1600 | 13 |
| A330I | 90000 | 8000 | 12000 | 130 |
| A330Y | 5000 | 2700 | 4000 | 100 |
| A330I/I332E | 3000 | 600 | 800 | 18 |
| V264I/I332E | 25800 | 1700 | 2000 | 8 |
| S239D/I332E | 1700 | 900 | 1100 | 11 |

YB2/0-derived CNTO 860 variants

| | | | | |
|---|---|---|---|---|
| WT | 12000 | 3000 | 4000 | 30 |
| I332E | 5000 | 700 | 600 | 7 |
| A330I | 25000 | 7000 | 8000 | 15 |
| A330Y | 4000 | 6000 | 6500 | 30 |
| A330I/I332E | 10000 | 3000 | 3000 | 7 |
| V264I/I332E | 3000 | 1600 | 6000 | 7 |
| S239D/I332E | 1900 | 6000 | 2800 | 7 |

### Summary of Experimental Findings in Examples 3 Through 5 Using the Variants Produced in Example 2:

Relative to CNTO 860, certain of the CNTO 860 sequence variants described here showed reduced affinity for the FcγRIIb inhibiting receptor, which could result in greater efficacy in vivo, since binding to the inhibiting receptor has been shown to reduce Ab efficacy. In particular, the A330I mutant expressed in YB2/0 cells has dramatically reduced (500×) binding to FcγRIIb while showing only moderately reduced (8×) binding to FcγRIIa, only slightly reduced (<2×) binding to FcγRI, and slightly increased (2-3×) activity in NK-mediated ADCC. Thus, the additive effects of amino acid mutations combined with low-fucose glycan yielded Ab variants more potent than the same Ab with either modification alone

The effects of combining the A330I mutation and the I332E mutation is a novel approach to Fc-engineering and yielded an Ab variant that maintains high affinity for FcγRI, showed approximately 10-fold enhanced activity in the ADCC assays described (related to FcγRIIIa affinity), but had substantially reduced affinity for the inhibiting receptor FcγRIIb. Such an FcγR binding profile may offer distinct advantages when macrophage-like cells are the relevant immune effector cell in vivo.

We claim:

1. An antibody variant of a parent antibody capable of binding tissue factor with a tissue factor binding region and neutralizing one or more biological activities of tissue factor, said antibody variant comprising an Fc-region capable of binding an Fc-receptor, wherein the Fc-region comprises at least one substitution in the Fc-region and the antibody variant demonstrates enhanced ADCC activity as compared to the parent antibody.

2. The antibody variant according to claim 1, wherein the ADCC activity is measured in a europium or chromium release ADCC assay.

3. The antibody variant according to claim 1 comprising a substitution in a human IgG1 Fc-region at position 330 or 332 selected from the group consisting of A330Y, A330I, and I332E.

4. The antibody variant according to claim 2 wherein the tissue factor binding region is derived from the TF8-5G9 antibody and the antibody variant comprises a human IgG1 Fc-region having at least one substitution from the parent antibody.

5. The antibody variant according to claim 1 wherein the tissue factor binding region comprises:
(a) a heavy chain variable region comprising residues 1-117 of SEQ ID NO: 2; and
(b) a light chain variable region comprising residues 1-108 of SEQ ID NO: 4, wherein the antibody variant has an Fc receptor binding region having higher affinity for the Fc receptor than the antibody CNTO 860.

6. The antibody variant according to claim 5, wherein the Fc receptor binding region is derived from an IgG1 antibody constant region and has a substitution selected from the group consisting of A330Y, A330I, and I332E wherein the I332E variant may optionally further comprise a second substitution selected from A330I, V264I, and S239D.

7. The antibody variant of claim 1, wherein the antibody is expressed in a host cell line characterized by Fc-region glycans with relatively low fucose content.

8. An isolated monoclonal antibody competing for binding to human TF with the monoclonal antibody variant of claim 1.

9. The antibody variant of claim 1, wherein the antibody variant is a human antibody.

10. An isolated human TF antibody, wherein the TF antibody:
(a) competes with CNTO 860 for binding to human tissue factor;
(b) has an affinity to TF on MDA-MB-231 human breast carcinoma cells as measured by flow cytometry equivalent to CNTO 860; and
(c) shows equivalent killing of HCT116 human colorectal carcinoma cells at a lower concentration than WT CNTO 860 in a chromium release ADCC assay.

11. A pharmaceutical composition comprising the antibody or antibody variant of claim 1 and a pharmaceutically acceptable carrier.

12. The composition according to claim 11, wherein the antibody or antibody variant is combined with an anti-neoplastic agent selected from the group consisting of a phosphotyrosine kinase (PTK) inhibitor, radiopharmaceutical, an estrogen receptor modulator, a retinoid, a topoisomererase inhibitor, a cytotoxin, an alkylating agent, a nitrogen mustard, a nitrosourea, an antimetabolite, a mitotic inhibitor, and a radiosensitizer.

13. The composition according to claim 12, wherein the anti-neoplastic agent is the PTK inhibitor erlotinib.

14. An immunoconjugate comprising the antibody according to claim 1 linked to a therapeutic agent.

15. The immunoconjugate of claim 14, wherein the therapeutic agent is a cytotoxin.

16. The immunoconjugate of claim 15, wherein the therapeutic agent is a radioisotope.

17. A pharmaceutical composition comprising the immunoconjugate of claim 14 and a pharmaceutically acceptable carrier.

18. An isolated nucleic acid molecule encoding the antibody or antibody variant of claim 1.

19. The isolated nucleic acid molecule of claim 18 wherein the nucleic acid molecule is incorporated into an expression vector.

20. An isolated nucleic acid molecule encoding at least one isolated anti-TF antibody having at least one variable region comprising residues 1-119 of SEQ ID NO: 2 or residues 1-108 of SEQ ID NO:4.

21. A transfectoma comprising the isolated nucleic acid of claim 18.

22. A prokaryotic or eukaryotic host cell comprising an isolated nucleic acid according to claim 18.

23. A host cell according to claim 22, wherein said host cell is at least one selected from COS-1, COS-7, HEK293, BHK21, CHO, BSC-1, Hep G2, 653, SP2/0, 293, HeLa, YB2/0, myeloma, lymphoma cells, Perc.6, or any derivative, immortalized or transformed cell thereof.

24. A method for producing an anti-TF antibody or antibody variant according to claim 1, comprising translating a nucleic acid encoding the antibody or variant under conditions in vitro, in vivo or in situ, such that the TF antibody or antibody variant is expressed in detectable or recoverable amounts.

25. A transgenic nonhuman animal expressing a human antibody or antibody variant according to claim 1.

26. A method of inhibiting growth of a cell expressing TF, comprising contacting the cell with an effective amount of an antibody or antibody variant according to claim 1 such that the growth of the cell is inhibited.

27. A method of treating or preventing a disease characterized by growth or metastasis of tumor cells, in a subject comprising administering to the subject at least one anti-TF agent selected from the group consisting of an antibody or antibody variant according to claim 1, a composition or immunoconjugate according to claim 11, a nucleic acid molecule according to claim 18, and host cells according to claim 23 in an amount effective to treat or prevent the disease.

28. The method of claim 27, wherein the disease is cancer.

29. The method of claim 27 wherein the anti-TF agent is administered in combination concurrently or sequentially with at least one other therapeutic agent.

30. A method according to claim 27, further comprising administering, prior, concurrently or after said anti-TF agent, at least one composition comprising an effective amount of at least one compound or protein selected from at least one of a detectable label or reporter, an anti-neoplastic agent, a TNF antagonist, an antirheumatic, a muscle relaxant, a narcotic, a non-steroid anti-inflammatory drug (NSAID), an analgesic, an anesthetic, a sedative, a local anesthetic, a neuromuscular blocker, an antimicrobial, an antipsoriatic, a corticosteriod, an anabolic steroid, an erythropoietin, an immunization, an immunoglobulin, an immunosuppressive, a growth hormone, a hormone antagonist, a reproductive hormone antagonist, a hormone release modulator, a hormone replacement drug, a signal transduction inhibitor, an apoptosis inducing agent, an antidepressant, an antipsychotic, a stimulant, an asthma medication, a beta agonist, an inhaled steroid, an epinephrine or analog, a cytokine, or a cytokine antagonist.

31. An anti-idiotype antibody or fragment that specifically binds at least one anti-TF antibody or antibody variant according to claim 1.

32. The method according to claim 27, in which the anti-TF agent competes with monoclonal antibody TF8-5G9 for binding to human TF.

33. The method according to claim 27, in which the anti-TF agent is administered intravenously, subcutaneously, intramuscularly, intranasally, subdermally, by inhalation, or orally.

34. The method according to claim 27, in which the anti-TF agent is administered in the amount of from 0.05 mg/kg to 12.0 mg/kg body weight.
